(19) United States
(12) Patent Application Publication
(10) Pub. No.: US 2021/0373009 A1
(43) Pub. Date: Dec. 2, 2021

(54) PLASMONIC META-SURFACE BASED MOLECULAR SENSORS AND METHODS FOR MAKING AND USING THEM

(71) Applicant: UNIVERSITY OF NORTH TEXAS, Denton, TX (US)

(72) Inventors: Arup Neogi, Denton, TX (US); Eric Ashalley, Cape Coast (GH)

(21) Appl. No.: 17/334,171

(22) Filed: May 28, 2021

Related U.S. Application Data
(60) Provisional application No. 63/031,517, filed on May 28, 2020.

(51) Int. Cl. G01N 33/543; G01N 33/569; C12Q 1/70; G01N 21/31; G06N 3/04

(52) U.S. Cl. CPC G01N 33/54373; G01N 33/56983; C12Q 1/70; G01N 2201/0636; G06N 3/04; G01N 2333/165; G01N 21/31

(57) ABSTRACT

A chiral metamaterial absorber modeled after the yin-yang symbol comprising: a top yin-yang shaped Au nanoparticles (YNPs); a PMMA layer; an Au backreflector; and a bottom glass layer. In alternative embodiments, provided are devices acting as sensors for detecting a nucleotide such as a cDNA and/or a protein such as a protein from a pathogen such as a bacteria or a virus, wherein the cDNA or protein can be derived from a Coronavirus, for example, a SARS CoV-2 or COVID-19 virus. In alternative embodiments, devices acting as sensors as provided herein can also be used to detect any protein for diagnostic or therapeutic purposes, wherein the protein can be derived from a blood or plasma sample, or a tissue sample, for example, a biopsy, from an individual in need thereof, for example, a human or an animal.

### CROSS-REFERENCE TO RELATED APPLICATIONS

This application claims priority to U.S. Provisional Patent Application Ser. No. 63/031,517, filed May 28, 2020, which application is incorporated by reference herein in its entirety.

### STATEMENT OF FEDERALLY SPONSORED RESEARCH

This invention was made with government support under Grant No. NSF 1741677 awarded by the National Science Foundation. The government has certain rights in the invention.

### FIELD

The present disclosure relates generally to biosensors and the detection of biological molecules. Example embodiments relate more particularly to chiral nanostructures and methods for the design and use of the same. In alternative embodiments, provided are methods for detection biological molecules in a sample derived from an individual in need thereof to detect the presence of any biological molecule, for example, a biological molecule derived from a pathogen such as a virus, or to aid in the diagnosis, and possible eventual treatment, of a disease, condition or an infection, for example, a viral infection such as a coronavirus infection, such as a COVID-19 or SARS-2 infection.

### BACKGROUND

Chiral nanostructures are non-superimposable to their mirror image, and they produce a different optical response for left circularly polarized (LCP) and right circularly polarized (RCP) light. This difference can be quantified by measuring chiroptical responses such as circular dichroism (CD) and chiral anisotropy factor (gCD or g-factor), providing metrics for the chiral asymmetry of these structures.

Limited by their small chiral asymmetry and electromagnetic interaction volume, naturally occurring chiral structures such as proteins formed by amino acids, DNA, and RNA exhibit low CD signals, which reduced detection efficiencies. This limitation can be resolved by coupling these naturally occurring structures to artificially engineered plasmonic nanostructures such as plasmonic metamaterials under resonant excitation. Plasmonic metamaterials can strongly enhance a molecular CD signal and even displace it to longer wavelengths due to the interaction of the strong resonant plasmonic near-field with the chiral biomolecules.

Chiral plasmonic metamaterials exhibit inherent CD and the geometry of the metamaterials can be used to control the degree of chiral asymmetry. The shape and metamaterial geometry can be used to design and manipulate nanoscale chiroptical effects. By changing the geometry of chiral and non-chiral plasmonic metamaterials, the resonant spectrum can be optimized to target specific applications using bio-assembled as well as non-biological systems. The interaction of biomolecular structures with super-chiral fields from chiral plasmonic nanostructures can also induce asymmetric changes in the retardation phase effect of the modes in the nanostructures and provide fingerprints for enantiomer discrimination. The range of applications for plasmonic chiral systems include, among others, improved spectroscopy techniques, photocurrent generation, and bolometry.

While chiral plasmonic metastructures offer very strong CD signals, they also present design and optimization challenges due, for instance, to the iterative and case-by-case simulations required to solve Maxwell's equations for a given geometry, which can be a time- and resource-intensive process. Given the complex relationship between chiral plasmonic metamaterials and their chiroptical response, a data-driven approach to the design and optimization of the structures can improve its efficiency.

Deep-learning (DL) is a data-driven technique for analysis and prediction that has permeated several disciplines such as natural language processing, image recognition, genetics, and biology. Instead of generating simulation results by running through predefined systems of equations in a given geometry, a DL architecture can be trained to recognize patterns in a given dataset, identify attributes, and predict responses, thanks to its capability to reproduce arbitrarily complex functions. As a type of representation or feature learning, deep-learning brings machine learning closer to artificial intelligence (AI) where human-like and exceedingly challenging tasks can be completed by trained systems. A typical example of the capabilities of this technique is the success of deep neural networks with reinforcement learning in playing games such as chess checkers or shogi.

Neural network models have more recently been utilized as a fast prototyping tool in the design of metamaterials. Generative adversarial network (GAN) and image processing approaches have also been used to link the geometry of metamaterials with their optical response. Hybrid techniques consolidating compositional pattern-producing networks and cooperative coevolution as well as those blending deep generative models with semi-supervised learning have also been proposed for the inverse design of metastructures. The prediction power of deep-learning models even extends to the near- and far-field distributions of arbitrary 3D nanostructures.

However, DL-based prediction models for metamaterial design currently suffer from low accuracy owing to the huge mismatch between the dimensions of input and output parameters, especially for inverse retrieval designs. Further, the generation of the training set via traditional simulation routes can be time and resource intensive for the study of nanoscale chirality, requiring an efficient use of the generated training set during the ML design and training. In addition, capturing the local optima (plasmonic/CD resonances) of chiroptical responses is challenging for most DL models and sometimes necessitates the setup of an auxiliary network, which adds to the complexity of the model.

### SUMMARY

According to some embodiments, a chiral metamaterial absorber modeled after the yin-yang symbol comprising: a top yin-yang shaped Au nanoparticles (YNPs); a PMMA layer; an Au backreflector; and a bottom glass layer is provided.

According to another aspect of the disclosed embodiments, a device comprises a top asymmetric Au metastructure comprising at least one Au “yin-yang-shaped” nanoantenna disposed on a substrate.

According to another aspect, a method for designing and/or optimizing a three-dimensional chiral metamaterial implemented by a processor and memory comprises:
receiving input geometric design parameters for the chiral metamaterial by an end-to-end functional bi-directional multitask deep-learning (MDL) model comprising a neural network, the MDL model being configured to predict a chiroptical response of the chiral metamaterial via a forward prediction path; and
receiving input for a chiroptical response by the MDL model, the MDL model being further configured to predict geometric design parameters for the chiral metamaterial via an inverse prediction path.

According to another aspect, a system for three-dimensional chiral metamaterial design and optimization comprises:
an end-to-end functional bi-directional deep-learning (DL) model implemented by a processor and memory, wherein the model utilizes multitask joint learning features to recognize, generalize and explore a relationship between the metamaterials' geometry and their chiroptical response in both forward and inverse directions,
wherein the model efficiently realizes both forward and inverse retrieval tasks.

According to another aspect, a chiral metamaterial absorber modeled after the yin-yang symbol comprises:
a top yin-yang shaped Au nanoparticle (YNP),
a PMMA layer,
an Au backreflector; and
a bottom glass layer.

According to another aspect, a MDL-optimized chiral structure is applied in the application of sensing biomolecular enantiomers.

On alternative embodiments, provided are devices comprising:

a top asymmetric Au metastructure comprising at least one Au “yin-yang-shaped” nanoantenna disposed on a substrate.

On alternative embodiments device as provided herein further comprise: a backreflector layer disposed between the top asymmetric Au metastructure and the substrate.

On alternative embodiments of devices as provided herein:
the device further comprises a polymer layer disposed between the top asymmetric Au metastructure and the backreflector layer;
the backreflector layer comprises gold;
the top asymmetric Au metastructure comprises at least two “ying-yang-shaped” nanoantennas separated by a gap, wherein the gap and the nanoantenna radii are optimized using a machine learning algorithm;
the top asymmetric Au metastructure comprises a central nanodisk with encompassing three “yin-yang-shaped” split-ring nanoantennas in clockwise direction wherein the central nanodisk and flanking nanoantenna radii are optimized using a machine learning algorithm;
an initial separation gap d, between the nanodisk and nanoantennas from their centers is optimized to induce maximum interparticle coupling; and/or
the polymer layer comprises PMMA, the backreflector layer comprises gold, and the deposited on a gold coated silicon substrate.

On alternative embodiments, provided are methods of using a device as provided herein, wherein circularly polarized light (CPL) is incident from a first side and reflected and transmitted lights are collected at the first side and an opposing side.

On alternative embodiments, provided are methods of fabricating a chiral structure as a plasmonic meta-surface on a transparent substrate using electron beam evaporation and electron beam lithography processes comprising:

preparing a transparent silicon dioxide substrate with a combination of Au/dielectric layers, wherein the structures comprise SiO₂ coated with about 2 nm of chromium for the adhesion of the reflective metallic mirror layer of about 100 nm on a substrate; and

covering an Au layer with about 30 nm Alumina (Al₂O₃), which has a slightly higher refractive index.

On alternative embodiments of methods as provided herein: the Au and dielectric layers are deposited using electron beam evaporation.

On alternative embodiments, provided are methods for designing and/or optimizing a three-dimensional chiral metamaterial, the method being implemented by a processor and memory, the method comprising:

receiving input geometric design parameters for the chiral metamaterial by an end-to-end functional bi-directional multitask deep-learning (MDL) model comprising a neural network, the MDL model being configured to predict a chiroptical response of the chiral metamaterial via a forward prediction path; and

receiving input for a chiroptical response by the MDL model, the MDL model being further configured to predict geometric design parameters for the chiral metamaterial via an inverse prediction path.

On alternative embodiments of methods as provided herein:
the MDL model is trained using a joint-learning feature based on a joint multitask cost function to enhance prediction accuracy for the forward and/or inverse prediction paths;
the MDL model is configured in the forward prediction path to perform at least a main task and an auxiliary task;
the MDL model is configured to normalize the input geometric design parameters and combine the normalized geometric design parameters with spectral data points;
the chiral metamaterial comprises at least one “yin-yang-shaped” nanoantenna disposed on a substrate,
the geometric design parameters comprise one or more of a substrate layer thickness, a backreflector layer thickness, a polymer layer thickness, radii of the “yin-yang-shaped” nanoantenna, or a gap between multiple “yin-yang-shaped” nanoantennas, and/or
the chiroptical response comprises one or more of left circularly polarized (LCP) light absorption, right circularly polarized (RCP) light absorption, or circular dichroism (CD) spectral values.

On alternative embodiments, provided are systems for three-dimensional chiral metamaterial design and optimization comprising:

an end-to-end functional bi-directional deep-learning (DL) model implemented by a processor and memory, wherein the model utilizes multitask joint learning features to recognize, generalize and explore a relationship between the metamaterials' geometry and their chiroptical response in both forward and inverse directions,

wherein the model efficiently realizes both forward and inverse retrieval tasks.

On alternative embodiments, provided is a chiral metamaterial absorber modeled after the yin-yang symbol comprising:

a top yin-yang shaped Au nanoparticle (YNP),

a PMMA layer,

an Au backreflector; and

a bottom glass layer.

On alternative embodiments, provided are MDL-optimized chiral structures applied in the application of sensing biomolecular enantiomers.

In alternative embodiments, provided are methods for detecting a biological molecule of interest in a sample comprising contacting a biological sample with a device or sensor as provided herein, and determining if the biological molecule of interest specifically binds to a nanoantenna of the device or sensor,

wherein optionally the biological molecule of interest is derived or taken from a blood, serum or sputum sample, or a tissue sample, or a biopsy, and optionally the biological molecule of interest is or comprises a nucleic acid, a protein, a lipid or a polysaccharide, and optionally the nucleic acid comprises a DNA, a cDNA or an RNA,

wherein the biological molecule of interest can specifically bind to a nanoantenna of the device or sensor, wherein the nanoantenna of the device or sensor has bound or conjugated to it a ligand for the biological molecule or a molecule or composition (which can be a small molecule, a nucleic acid, a protein, a lipid, a saccharide or a polysaccharide) that is capable of specifically binding to the biological molecule,

and optionally the nanoantenna comprises or has affixed thereon a biological molecule, and optionally the biological molecule comprises a nucleic acid, a protein, a lipid or a polysaccharide, and optionally the nucleic acid comprises a DNA, a cDNA or an RNA,

optionally the biological molecule of interest is derived or taken from a pathogen, and optionally the pathogen is a bacteria or a virus, and optionally the pathogen is a coronavirus, and optionally the coronavirus is a SARS-2 or COVID-19 virus.

In alternative embodiments, the methods further comprise diagnosing a disease, infection or condition comprising: contacting a biological sample with a device or sensor as provided herein, and determining if the biological molecule of interest specifically binds to a nanoantenna of the device or sensor, wherein if the biological molecule of interest is detected or determined to be present in the biological sample by the detection of its specific binding to a nanoantenna of the device or sensor (wherein the biological molecule of interest specifically binds to a nanoantenna of the device or sensor, wherein the nanoantenna of the device or sensor has bound or conjugated to it a ligand for the biological molecule or a molecule or composition (which can be a small molecule, a nucleic acid, a protein, a lipid, a saccharide or a polysaccharide) that is capable of specifically binding to the biological molecule), an individual in need thereof from which the biological sample was derived is diagnosed with the disease, condition or infection, wherein optionally the infection is a viral infection, and optionally the viral infection is a coronavirus infection, and optionally the coronavirus infection is a COVID-19 or a SARS-2 infection.

In alternative embodiments, the methods further comprise treating or ameliorating the disease, infection or condition comprising: administering a treatment or a drug to treat or ameliorate the disease, infection or condition diagnosed or detected in the individual in need thereof.

In alternative embodiments, provided are methods for isolating or separating (or substantially isolating or separating) biological molecules from a biological sample comprising contacting a device or sensor as provided herein with the biological sample under conditions wherein a biological molecule of interest specifically binds to a nanoantenna of the device or sensor, wherein the nanoantenna of the device or sensor has bound or conjugated to it a ligand for the biological molecule or a molecule or composition that is capable of specifically binding to the biological molecule.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

Other features and advantages of the invention will be apparent from the following specification taken in conjunction with the following drawings.

### DESCRIPTION OF THE DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

The present disclosure will become more fully understood from the detailed description and the accompanying drawings, wherein:

FIG. 1A illustrates a single yin-yang shaped Au nanoparticles (YNP) chiral meta-absorber array with definition of incident circularly polarized lights and unit cell dimensions according to some embodiments.

FIGS. 1B-1C. FIG. 1B illustrates three example single YNP metastructure configurations: Au YNP/Glass (YG); Au YNP/PMMA/Glass (YPG); and Au YNP/PMMA/Au/Glass (YPAG). FIG. 10 illustrates absorption and circular dichroism (CD) spectra of the three metastructure configurations (YG, YPG and YPAG) in FIG. 1B, showing their plasmonic resonances λₚ (λₚ=620 nm, 625 nm, and 645 nm respectively), illustrating a strong chiroptical response for the metamaterial absorber case (YPAG), where R₀=100 nm, t₁=40 nm, t₂=50 nm, t₃=100 nm, and t₄=200 nm.

FIG. 1D shows the electric field and surface charge density distributions of the three example single YNP metastructure configurations of FIG. 1B according to example embodiments: YNP/Glass (YG), YNP/PMMA/Glass (YPG) and YNP/PMMA/Au/Glass (YPAG) at their plasmonic resonances, showing large enhancement (comparatively high CD signals) for the example metamaterial absorber case. The cut plane is through point A, the unit cell center (FIG. 1A). R₀=100 nm, t₁=40 nm, t₂=50 nm, t₃=100 nm, and t₄=100 nm.

FIGS. 2A-2H show optical and chiroptical properties including absorption and reflection spectra of example chiral metamaterial absorbers with 10 nm tip rounding radius according to example embodiments, including: (A) Absorption and (B) Reflection spectra of single yin-yang metamaterial absorber at varying polymer thicknesses; corresponding (C) CD and (D) gCD from (a) exhibiting a strong peak in the visible regime (around 635 nm wavelength) CD response from three gapped configurations of the chiral metamaterial absorber; (E) dimer with varying gap length d; (F) circular with varying PMMA thickness; (G) circular with central disk and varying central disk radius r; and (H) charge density distribution at the CD maxima (Peak A) for the 60 nm PMMA thickness in (C).

FIG. 3 is a schematic of a bi-directional multitask deep-learning model (MDL model) for a chiral metamaterial design (example geometry shown in inset) including forward design (FDP) and inverse design paths (IDP) according to example embodiments.

FIGS. 4A-4D illustrate multitask deep-learning (MDL) model performance and MDL-predicted CD progressions in accord with aspects of example embodiments.

FIGS. 4E-4G. FIG. 4E shows an example learning curve within 3000 epochs, illustrating the fast convergence of the MDL model of FIGS. 4A-4D in accord with aspects of example embodiments. FIG. 4F shows discretized model performance at selected t₁={60 nm, 80 nm, 100 nm} corresponding to the horizontal dots in FIG. 4C and FIG. 4D. FIG. 4G shows model performance at λ₀=755 nm across varying t₁, corresponding to the vertical short dashes through FIGS. 4C-4D, where R₀=100 nm, d=100 nm, t₂=50 nm and t₃=100 nm.

FIG. 4H is a map plot illustrating MDL-predicted CD progressions, including panels (a)-(d).

FIG. 41 shows example MDL-predicted CD progressions including CD evolution when varying PMMA thickness at t₁={30 nm, 60 nm, 90 nm} for R₀=100 nm.

FIGS. 5A-5D illustrate an inverse design path with an MDL model according to example embodiments, where FIGS. 5A-5B show simulated (green solid lines) and predicted (red dots) CD spectra, and FIGS. 5C-5D show corresponding simulated (green bars) and retrieved (red bars) geometric parameters.

FIGS. 5E-5G show inverse design plots for simulated and MDL-retrieved absorption spectra comparison for the systems providing the data in (e) FIG. 5A and (f) FIG. 5B. FIG. 5G shows a comparison between the CD values obtained with different variations of the ML techniques: separately training LCP and RCP spectra (black dashes); training only CD without auxiliary tasks (red dashes); training by an example MDL model (blue dashes); and the ground truth (pink solid line). The inset is a zoomed-in image at the resonances.

FIGS. 6A-6D. FIG. 6A shows weak CD resonant peak of a molecular enantiomer pair in the UV according to example embodiments. FIG. 6B illustrates a distinguishable dielectric medium-induced CD redshift according to some embodiments. FIG. 6C illustrates how the CD signal of the chiral molecules shifts its peak to the visible range according to example embodiments. FIG. 6D compares three variables for the left-handed chiral metamaterial absorber (LHcma) and right-handed chiral metamaterial absorber (RHcma) systems, under the dielectric chiral medium, according to example embodiments.

FIG. 7 illustrates the three-dimensional structure of SARS-CoV-2.

FIGS. 8A-8C illustrate: (A) schematic of an example triskelion-based (TNQ-based) chiral meta-absorber array with definition of incident circularly polarized lights and a unit cell with its dimensions; (B) three example metastructure configurations: TNQ/Glass, TNQ/PMMA/Glass and TNQ/PMMA/Au/Glass, and (C) a comparison of the surface electric field distribution maps for the TNQ/Glass metastructure at selected wavelengths upon left circularly polarized (LCP) (first row) and right circularly polarized (RCP) (second row) incidence revealing differences in field patterns according to some embodiments.

FIG. 9 illustrates a chiral-sensor chip according to example embodiments.

FIG. 10 illustrates a (large) difference in the circular dichroism (CD) induced by chiral triskelion structures according to example embodiments.

FIGS. 11A-11C illustrate example sequences for attaching to gold plasmonic chiral nanostructures such as nucleic acids (for example, viral nucleic acids) using thiol chemistry according to example embodiments.

FIG. 12 illustrates absorption data of a synthetic oligomer attached to the gold triskelion meta-surfaces on a quartz substrate light according to example embodiments.

### DETAILED DESCRIPTION

The field of chiral plasmonics has registered considerable progress with Machine-Learning (ML)-mediated metamaterial prototyping, drawing from the success of ML frameworks in other applications such as pattern and image recognition. Example embodiments herein provide, among other things, an end-to-end functional bi-directional deep-learning (DL) model for three-dimensional chiral metamaterial design and optimization. An example DL model utilizes multitask joint learning features to recognize, generalize, and explore in detail the non-trivial relationship between the metamaterials' geometry and their chiroptical response, avoiding the need for auxiliary networks or equivalent approaches to stabilize the physically relevant output.

Example DL models can efficiently realize both forward and inverse retrieval tasks with great precision, providing a beneficial tool for iterative computational design tasks in complex physical systems. Experiments illustrate behavior of sample ML-optimized structures in assisting the sensing of biomolecular enantiomers. Other example applications of provided metastructures include, but are not limited to, photodetectors, polarization-resolved imaging, and CD spectroscopy. Example ML frameworks provided herein are generally applicable to addressing various physical problems.

According to some embodiments, an end-to-end multitask deep-learning (MDL)-based model is provided for the design and optimization of three-dimensional chiral metamaterials. MDL models have gained root in the computational study of semantics, transportation, as well as pose and action recognition. Multitask learning draws on its implicit data augmentation, eavesdropping, attention focusing, representation bias, and regularization for effective and efficient generalization, eliminating the need for auxiliary networks or equivalent approaches to stabilize the model's output of physically relevant information.

Highly portable and functional multitask deep-learning (MDL) models can be provided in example embodiments to comprehensively study three-dimensional, arbitrarily complex chiral metamaterials. Usage of example MDL models can be exemplified with a chiral metamaterial designed after the yin-yang symbol.

Three-dimensional chiral metamaterial absorber structures (meta-structures) are provided according to example embodiments. These meta-structures may be designed and/or optimized using example MDL models. Some example meta-structures include nanoparticles shaped geometrically based on the yin-yang symbol, which are referred to as yin-yang nanoparticles (YNPs). Single or multiple YNPs can be provided, and these can be disposed on one or more layers or substrates. Multiple YNPs can be arranged on layers or substrates to provide various meta-structures. Example arrangements of multiple YNPs include but are not limited to dimers and circular arrangements. Some example circular arrangements surround a central disc to provide a triskelion structure (TNQ).

Example MDL models can be composed by a single end-to-end bidirectional architecture that is capable of performing optimization and inverse retrieval operations and takes advantage of the supporting role of two auxiliary tasks to facilitate the learning of a primary task, such as CD. This novel bidirectional architecture can provide a reduction of the complexity of the deep-learning framework while ensuring an efficient use of the training set towards a highly generalized system.

According to some embodiments, an MDL model for designing three-dimensional chiral metamaterials comprises a single bidirectional neural network solving two tasks: the accurate prediction of the full chiroptical response of a chiral metamaterial from a set of geometric parameters, via a forward prediction path; and the accurate retrieval of the geometric parameters that can produce a given input of a full chiroptical response, by solving the inverse problem, via an inverse prediction path.

To bridge the mismatch gap and enhance the prediction accuracy for both forward and inverse predictions, especially at CD and plasmonic resonances, a joint-learning feature may be incorporated in the model training. This feature ensures the comparison of errors in the learning of tasks, allowing fora well-generalized system. Consequently, example MDL models can ensure an efficient use of the training set to achieve faster convergence, providing practical systems and methods for implementing similar ML systems in a variety of design problems with nanotechnological applications.

As a data-driven approach, example MDL models can employ a dataset, e.g., a prior database, of results created with methods such as FEM simulations and/or experimental data. Such an approach helps avoid the huge computational cost that would be required to explore the vast design space of the physical system in fine detail. An additional advantage resides in the fact that a trained MDL model is a fast, lightweight, highly-transferrable tool that can drastically reduce the computational time used for subsequent studies of the associated system, even by others.

In experiments, given a set of geometric parameters, the forward design path of example MDL models predicted CD spectra with values virtually identical to the simulations used as ground truth. And, vice versa, for input CD spectra, the example MDL model retrieved the set of geometric parameters that would produce such input CD spectra by solving the inverse problem. As a result, trained example models can be used to explore the entire design space and thus render a complete account of the intricate relationship between the metamaterial's geometric parameters and its chiroptical response. This is made possible at least by the joint-learning feature incorporated in example models.

For nanophotonic applications, the design and prototyping process needs to be robust due to the complexity of light-matter interaction with non-trivial geometries. Multitasking deep-learning-based prediction models in example embodiments can aid in engineering any potential fabrication of the nanophotonic structures for desired optical and chiroptical response towards a variety of applications.

Example embodiments described in more detail Illustrate additional chiroptical properties of an example chiral metamaterial absorber structure in the context of its interaction with molecular enantiomers. The high efficiency and accuracy of the example end-to-end MDL model makes it a valuable tool for the study of complex physical phenomena, particularly for the design and prototyping of nanophotonic structures towards their application as biosensors, photodetectors, or in polarization-resolved imaging and Circular Dichroism (CD) spectroscopy, among others.

According to some embodiments, example MDL models can be employed in a system which can detect chiral molecules and other homochiral molecules with very high sensitivity. Using example models, the system can be tuned over a very large wavelength range by a deep learning (DL) process.

According to some embodiments, the machine learning described herein is employed to design novel plasmonic structures such as a yin-yang shaped chiral structure. According to some embodiments, the machine learning described herein is employed to evaluate novel designs of chiral plasmonic structures. According to some embodiments, the machine learning described herein is employed to design novel plasmonic structures such as a yin-yang shaped chiral structure and to evaluate novel designs of chiral plasmonic structures.

The artificial intelligence (AI) structure of example models can be used to design different configurations of example chiral meta-structures. According to some embodiments, sensing techniques based on the sensitivity of the rotation of light due to molecules to be detected are employed. According to some embodiments, the changes in the rotation caused by molecules to be detected are very weak and are not very sensitive as the wavelength of light is significantly longer than the dimension of the chiral molecules.

According to some embodiments, chiral plasmonic structures can be chosen so that the interaction of light with the small molecule is enhanced due to nanophotonics or the operation of the device in the nanoscale. This interaction of the light with the molecules at a given range of optical color can be enhanced by the AI model.

According to some embodiments, spiral structures are employed with Yin-Yang structures being a special class of these spiral-shaped chiral structures. Experimentally and theoretically, a number of chiral geometries have been investigated and demonstrate huge circular dichroism signals including varying configurations of gammadions, helices, and spirals.

According to some embodiments, example AI-based models can be employed to design structures like the yin-yang shaped structures described above or other structure, and designed yin-yang and other structures may be used, for instance, in sensors to detect small molecules.

According to some embodiments, device fabrication and experiments can be performed on clockwise and counter-clockwise Yin-yang structures. According to some embodiments, Yin-yang structures can create chiral photons (or circularly polarized photons in the nanoscale limit<200 nm in dimension). Such structures can also have a large meta-surface which acts as a very efficient absorber to increase the interaction of light with photons. This can lead to sensors with a very strong differentiation of the light when the Yin-yang structure interacts with a molecule to be detected. Such Yin-yang structures sensor can be made more sensitive than prior sensors.

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspects of the invention to the embodiments illustrated.

Preferred embodiments will now be discussed with respect to the drawings. The drawings include schematic figures that are not to scale, which will be fully understood by skilled artisans with reference to the accompanying description. Features may be exaggerated for purposes of illustration. From the preferred embodiments, artisans will recognize additional features and broader aspects of the invention.

Example Chiral Metamaterial Model

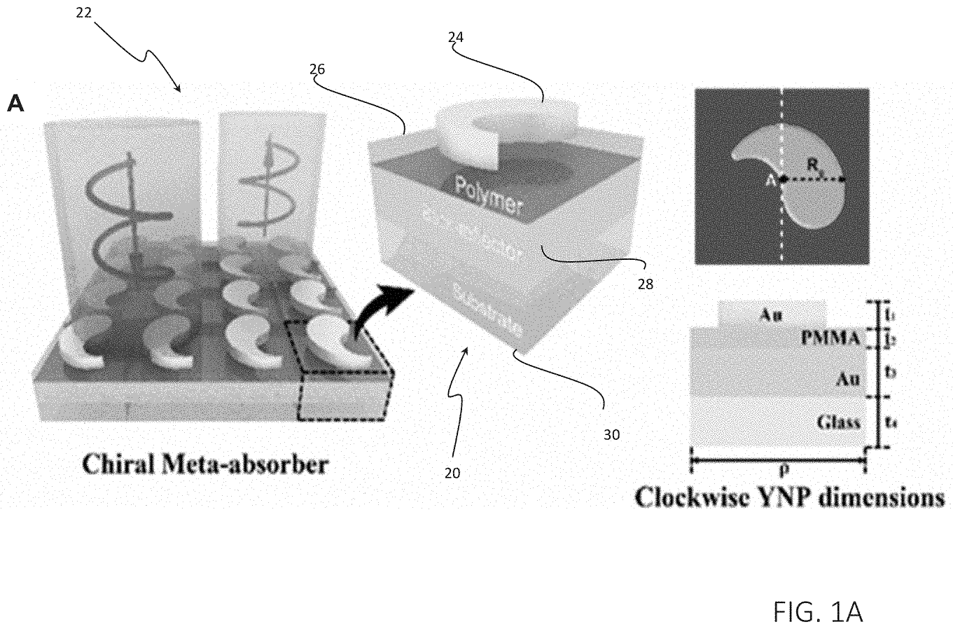

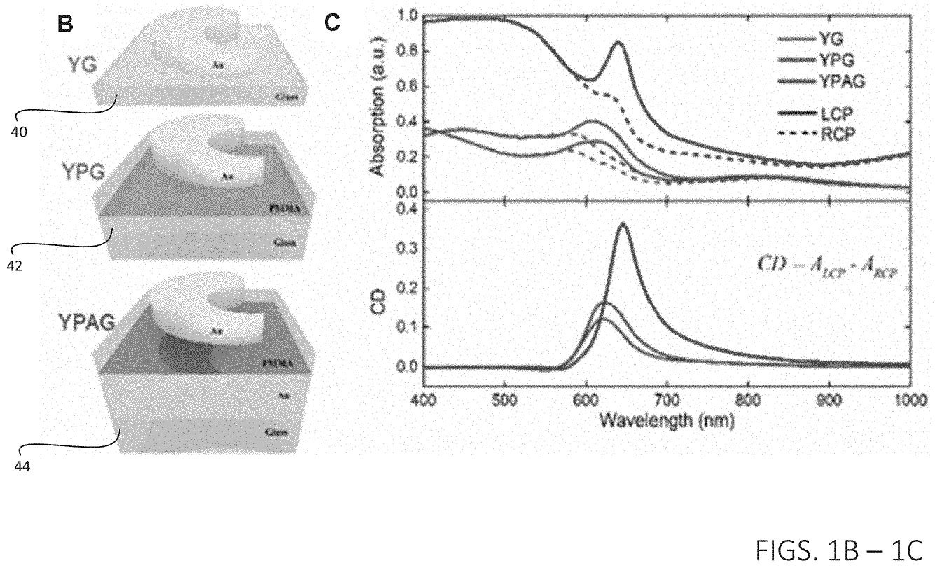

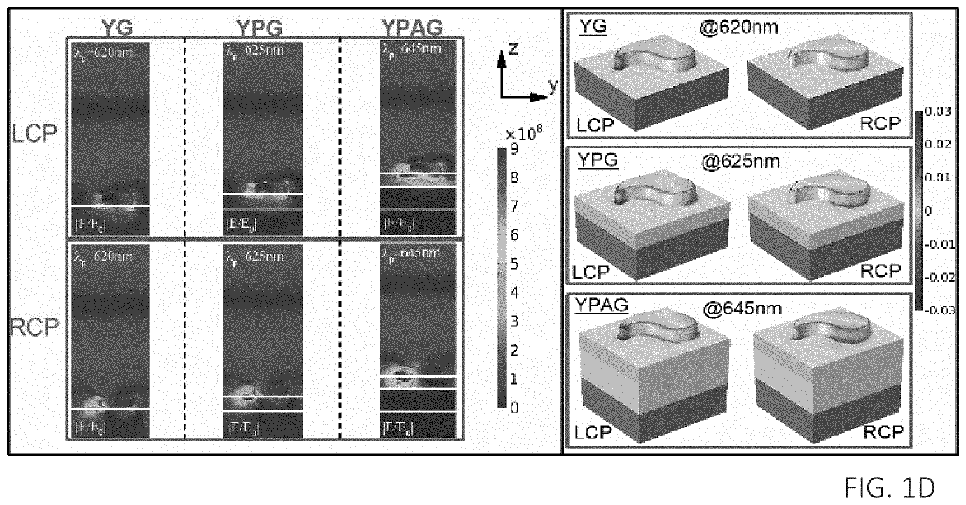

Example chiral metamaterial models include yin-yang shaped structures, and thus example models can be based geometrically on the yin-yang symbol. FIG. 1A shows a unit cell 20 of an example metamaterial absorber structure (meta-absorber structure) 22, as well as a single YNP chiral meta-absorber array with definition of incident circularly polarized lights. The example meta-absorber structure 22 includes top yin-yang shaped gold (Au) nanoparticles (YNPs) 24, a polymethyl methacrylate (PMMA) layer 26, an Au (or other suitable material) backreflector 28, and a bottom glass substrate layer 30.

The YNPs 24 can be defined by their radius R₀ and thickness t₁. An example period, p, is set following 2R₀+50 nm. To eliminate spurious effects due to mathematically sharp edges, the top corners and the tip of the YNP 24 can be rounded with a minimum radius, e.g., of 10 nm, corresponding to a R₀ of 100 nm, and scales up with larger values of R₀ to preserve the YNP shape.

Example dielectric constants of glass and PMMA are 2.13 and 2.25 respectively. The dielectric constants of the Au YNP 24 and the backreflector 28 can be interpolated from the Johnson and Christy dataset. The medium surrounding the example metastructure 22 can be chosen to be air, for instance.

In an example operation, an incident beam is normal to the metamaterial. Example models used in some experiments were calculated using a finite element method (FEM)-based commercial software package, COMSOL Multiphysics.

To illustrate the relevance of the example multilayered structure on the intensity of the fields and chiroptical response under resonance, FIG. 1B shows single YNP metastructure configurations under three structures of comparable dimensions: YNP/Glass (YG) 40, YNP/PMMA/Glass (YPG) 42, and YNP/PMMA/Au/Glass (YPAG) 44. The example YG setup 40 includes a 40 nm thick Au YNP and a glass substrate 30 with thickness t₄. For the YPG configuration 42, a PMMA polymer layer 26 with thickness t₂ is introduced on the glass substrate to create a YNP/PMMA interface. Fabrication of the homopolymer layer can be conveniently achieved by spin-coating. The third configuration (YPAG) 44 adds an Au back-reflector layer 28 of thickness t₃, between the PMMA 26 and glass layers 30. Such a structural combination with a backreflector largely increases the optical absorption of the system, by allowing the interaction between the nanoantenna and reflected modes.

FIG. 10 compares the absorption and circular dichroism (CD) spectra of the example YG, YPG and YPAG metastructures 40, 42, 44, showing their plasmonic resonances λₚ (λₚ=620 nm, 625 nm, and 645 nm respectively) and revealing a strong chiroptical response for the metamaterial absorber case (YPAG) 44. Here, R₀=100 nm, t₁=40 nm, t₂=50 nm, t₃=100 nm, and t₄=200 nm. The YPAG structure 44 shows a comparatively large and broad absorption peak and large differential absorption, both arising from the interaction between the Au YNP and the Au backreflector.

Additional comparison of the cross-sectional local field enhancement and surface charge density distributions for the three example structures 40, 42, 44 reveals sharp differences for left and right circularly polarized beams (LCP and RCP) illumination at their respective plasmonic resonances λₚ. This further illustrates the strong chirality of example YNP-based structures especially, but not necessarily, in an example meta-absorber case where the field is highly enhanced in the polymer spacer layer.

For chiral structures, the circular dichroism and g-factor are the prominent parameters that describe their chiroptical properties. These properties reflect inherent topological characteristics of chiral matter at the nano/micro scale.

The CD and g-factor (gCD) in the absorption can be respectively calculated from:

$$CD = {A}_{LCP} - {A}_{RCP} , {g}_{CD} = \frac{{A}_{LCP} - {A}_{RCP}}{\frac{[ {A}_{LCP} + {A}_{RCP} ]}{2}}$$

where A_{LCP} and A_{RCP} are the absorbance of the chiral nanostructure illuminated by left and right circularly polarized beams, respectively.

FIG. 1D shows electric field and surface charge density distributions of three single YNP metastructure configurations: YNP/Glass (YG), YNP/PMMA/Glass (YPG), and YNP/PMMA/Au/Glass (YPAG) at their plasmonic resonances, showing large enhancement (comparatively high CD signals) for the example metamaterial absorber case. The cut plane is through point A, the unit cell center (e.g., unit cell 20 in FIG. 1A). In this example, R₀=100 nm, t₁=40 nm, t₂=50 nm, t₃=100 nm, and t₄=100 nm.

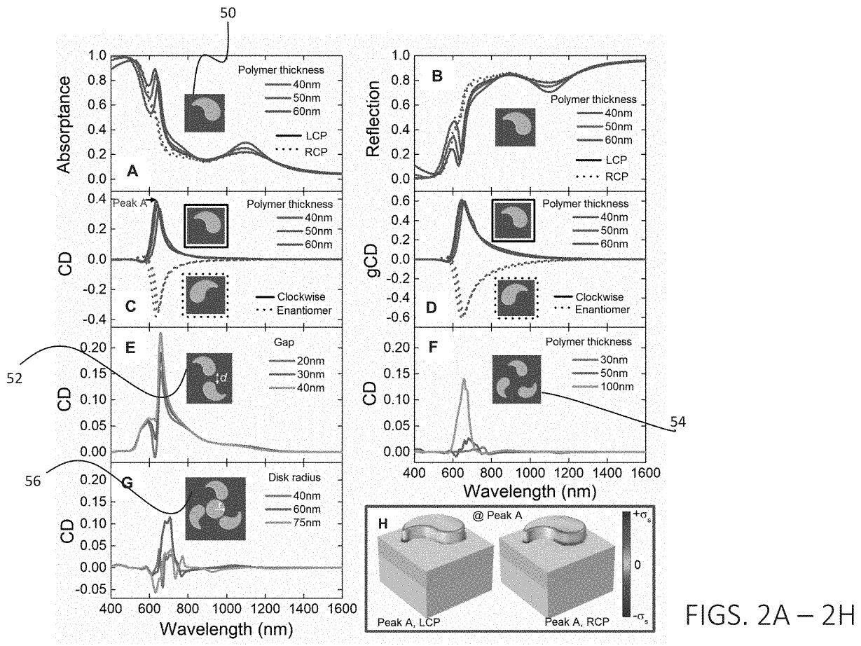

FIGS. 2(a)-(h) show CD and gCD calculations of an example chiral meta-absorber with 10 nm tip rounding radius and its variations. For example, FIG. 2A shows the absorption spectra of a single chiral YNP metamaterial absorber 50 at varying polymer thickness upon interaction with LCP and RCP light, with plasmonic peaks in the visible and near-IR range. FIG. 2B shows the corresponding complementary reflection spectra. Due to the effect of the Au backreflector, there is an enhanced differential absorption between LCP and RCP light. There is also an inversion in the CD response of the metamaterial absorber for YNP enantiomer pairs.

Hence, the handedness of the meta-structure is dictated by the handedness of the top chiral meta-atom. The CD and gCD responses of the single YNP metamaterial absorber 50 from FIG. 2A exhibiting a strong peak in the visible regime (around 635 nm wavelength) are illustrated in FIGS. 2C-2D. FIG. 2H shows the surface charge density distribution of the single YNP meta-absorber 50 corresponding to peak A in FIG. 2C, for LCP and RCP light.

Exploring other geometric arrangements of the example structure reveals additional significant properties. For a dimer, reducing the separation gap d increases the interaction of the two antennas and separates the CD signal in two distinct peaks, a feature that can be adopted for example sensing applications. FIG. 2E shows a dimer structure 52 with varying gap length.

For a circular arrangement 54 of the YNPs, e.g., of three YNPs (trimer), an interesting dependence of the magnitude of the CD response on the PMMA thickness is observed, with the CD almost vanishing for a PMMA thickness of 100 nm (FIG. 2F). With the introduction of a central disk, however, such as in triskelion (TNQ) structure 56, the plasmonic coupling between the YNP and the central disk can regulate the CD signal (FIG. 2G). That is, at a critical coupling distance, maximum CD can be achieved, and vice-versa, depending on the central disk's radius r.

Deep-Learning Model for Chiral Metamaterial Design

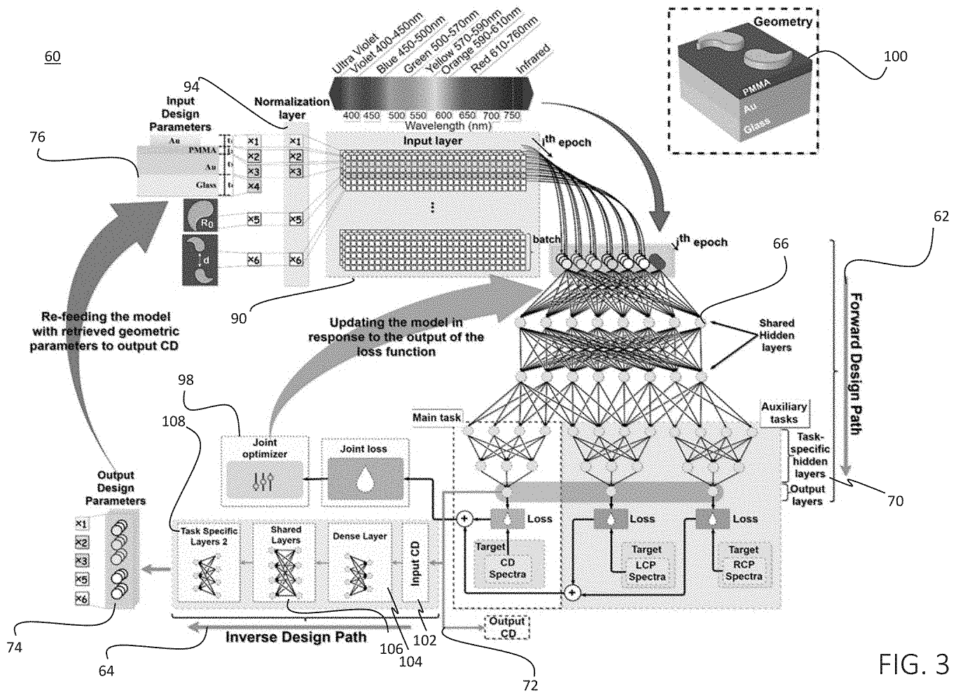

FIG. 3 illustrates an example bi-directional multitask deep-learning (MDL) model 60 for chiral metamaterial design. The example MDL model 60 includes forward design (FDP) 62 and inverse design 64 paths (IDP). Each path 62, 64 is composed by shared layers 66 and task specific layers 70 with joint optimization functionality. The example model 60 is setup in an end-to-end fashion, where the geometric design parameters, CD, and LCP/RCP absorption spectra, can be treated as input or output at specific ports 72, 74, 76.

Example geometric design parameters for the MDL model 60 can include, but are not limited to, the YNPs thickness, PMMA thickness, YNPs radius, and YNPs, which can be respectively represented as xᵢ where i=1, 2, . . . n (in an example model n=6). In an MDL model used in some example experiments x4 was taken as a constant, as explained below, but it is provided herein in an example general model to represent the general parametrization of example systems. The inset of FIG. 3 shows the metamaterial absorber geometry 52 used to exemplify the use of the example MDL model 60.

The enhanced chiral field and CD response in example metamaterial absorbers makes it ideally competitive for chirality-related applications. A base YNP structure can also be modified to generate other complex chiral designs, e.g., dimers and trimers (see FIG. 2).

However, the optical response of such structures can be highly sensitive to small variations of their geometric parameters. Thus, the optimization of a given design involves mapping out the response of many configurations across the design space. Each of these combinations requires solving the full electrodynamic problem under the illumination conditions, which is computationally costly.

In example methods, by combining a relatively small set of full wave simulations with an example MDL approach, the design space can be explored in fine detail at a greatly reduced computational cost. Various planar chiral geometries have been investigated and shown to demonstrate huge CD signals including spirals, gammadions, L-shaped, r-shaped, and z-shaped nanostructures.

For illustrating example MDL models, a variation of the above YNP metastructure defined by double top YNPs with radius, thickness and gap distance R₀, t₁, and d respectively (FIG. 3) is adopted. The polymer thickness, t₂, and the Au backreflector thickness, t₃, affect the reflection of the beam and its interaction with the chiral resonance of the YNP, regulating the shift in the CD spectra. Due to the dominant effect of the Au backreflector, the variation of thickness of the glass layer, t₄, was assigned a fixed value of 400 nm in an example method. The period of the structures is set following:

Pₓ=3R₀+50 nm,P_{y}=2(d+R₀+25 nm)

Here, Pₓ is the period along the x-direction and P_{y} is the period along the y-direction. By considering the double YNP structure, the gap between the nanoresonators as an additional parameter influencing the CD response is introduced, which is in a highly non-trivial relationship with the other geometric parameters.

This example structure was chosen to illustrate an example MDL model. The continuous curved surfaces and the difference in scale between the whole structure and its finest features, such as its tip, increase the complexity of the numerical optical simulation of the metamaterial, thus representing a desirable candidate for alternative or supporting computational approaches. Further, the complex non-linear relationship between the different design parameters and the structure's optical response makes it a good example to illustrate the power of an example MDL approach. However, it will be appreciated that the MDL approach is likewise applicable to other structures, including example structures shown and described herein.

The example MDL model 60 shown in FIG. 3 is a bidirectional neural network that includes an input layer 90, tensor layers, a normalization layer 94, optical and chiroptical task execution layers 70, and an optimization layer 98. The inset in the top right of FIG. 3 shows the schematic of an example metamaterial absorber 100 used to train the MDL model. The example MDL model 60 considers the plasmon resonant peaks with a regression learning objective. Example MDL models provide unique multitasking and joint learning capabilities that contribute to the efficient generalization of the parameter space.

Forward design: In the example forward prediction path 62, the varying dataset scales across the input design parameters, absorption, and CD responses. This may make the model data-scale-dependent if trained directly, resulting in poor generalization. To eliminate the effect of the varying input length scales on the generalization of the model, an example MDL model can employ a normalization layer 94 following the relation:

$${X}_{norm , ( a , b )} = \frac{{X}_{( a , b )} - {\overline{X}}_{b}}{σ ( {X}_{b} )} \qquad ( 1 )$$

where a and b index the row and column respectively such that X_{b} is the b^{th} column of the input parameter matrix, X. \tilde{X}_{b} and σ(X_{b}) are the mean and standard deviation, respectively.

The example normalization provides a well-conditioned dataset for optimization by ensuring that the training is less sensitive to the scale of features to be processed by the shared layer 66. At the shared layer 66, an example model adopts a hard parameter sharing of four hidden layers, each with 1024 nodes. These hidden layers are shared between all the individual task-specific output layers of the network. The shared hidden layers 66 hold computational weights from the task-specific layers 70. That is, the CD learning can leverage the LCP/RCP spectra task-specific learning to enhance accuracy. All hidden layers can be regularized by applying penalties on layer activity during optimization, e.g., with an £₂ regularizer, in order to learn sparse features and internal representations of raw observations.

The task-specific layer 70 includes two independent parts; the main task and auxiliary tasks. The auxiliary tasks may be, for instance, sub-tasks expected to assist in finding rigorous, rich, and robust representation of the input design parameters to benefit the main task. Learning auxiliary tasks restricts the parameter space during optimization and pushes for a faster convergence. The main task, which characterizes the desired output response, exploits and jointly learns from the auxiliary tasks via the shared layer 66. The main and auxiliary tasks correspondingly generate three single-task losses.

To optimize the MDL network, a joint multitask cost function comprising the three single-task losses can be minimized. For instance, Let i index the training set and j the dependent variables for three tasks, which in this example are the LCP, RCP, and CD learning tasks. Assuming these three tasks, with realizations yᵢⱼ, are independent and conditional to the prediction returned by a model ƒ, with adjustable parameters w (the weight), on input xᵢ, and that the error is normally distributed and zero-mean, with variance σⱼ², which depends only on j (the task), the log-likelihood function can be written as follows:

$${Σ}_{ij} log ( \frac{1}{\sqrt{2 {πσ}_{j}}} exp ( - \frac{{( {y}_{ji} - {f}_{j} ( {x}_{i} ; w ) )}^{2}}{2 {σ}_{j}^{2}} ) )$$

Applying the basic properties of the log function, the above equation becomes:

$${Σ}_{ij} ( - log ( \sqrt{2 π} ) - \frac{1}{2} log {σ}_{j}^{2} - \frac{{( {y}_{ji} - {f}_{j} ( {x}_{i} ; w ) )}^{2}}{2 {σ}_{j}^{2}} )$$

Looking for stationary points of this loss with respect to the variance σⱼ², and dropping the constant additive term, one has:

$$\frac{\partial }{\partial {σ}_{j}^{2}} {Σ}_{i} ( - \frac{1}{2} log {σ}_{j}^{2} - \frac{{( {y}_{ji} - {f}_{j} ( {x}_{i} ; w ) )}^{2}}{2 {σ}_{j}^{2}} ) = 0$$

From the linearity of partial derivatives, and simplifying the derivatives for each term, one has:

$${Σ}_{i} ( - \frac{1}{2 {σ}_{j}^{2}} \frac{{( {y}_{ji} - {f}_{j} ( {x}_{i} ; w ) )}^{2}}{2 {σ}_{j}^{4}} ) = 0$$

Extracting the first term from the summation gives:

$$- \frac{N}{2 {σ}_{j}^{2}} + {Σ}_{i} ( \frac{{( {y}_{ji} - {f}_{j} ( {x}_{i} ; w ) )}^{2}}{2 {σ}_{j}^{4}} ) = 0$$

Here, N is the size of the training set. Further simplification yields:

$$- N + {Σ}_{i} ( \frac{{( {y}_{ji} - {f}_{j} ( {x}_{i} ; w ) )}^{2}}{2 {σ}_{j}^{2}} ) = 0$$

$${σ}_{j}^{2} = \frac{1}{N} {Σ}_{i} {( {y}_{ji} - {f}_{i} ( {x}_{i} ; w ) )}^{2}$$

Substituting into the log density function and simplifying provides:

−Σⱼ log(Σᵢ(yⱼᵢ−ƒⱼ(xᵢ;w))²)

By changing the signs and exponentiating to get back to the original form of the quadratic loss, the principal multi-task cost function, subject to optimization, can be expressed as:

Πⱼ(Σᵢ(yⱼᵢ−ƒⱼ(xᵢ;w))²) (2)

In equation (2), i and j index the training set and the three learning tasks respectively, and yⱼᵢ refers to the simulated outputs from the three tasks (CD and LCP/RCP absorption signals). The model function, ƒ, takes as input xᵢ, which in an example method is the 1x5 design parameter matrix comprising the YNP radius R₀, the gap distanced, the YNP thickness t₁, the polymer thickness t₂, and the Au backreflector thickness t₃ with weight, w. The definition of d is illustrated in FIG. 3 (top left).

In an example optimization, the mean squared error (MSE), the quadratic loss which is the sum of squared distances between the target variable (simulated CD) and predicted CD values, is adopted to average the losses over the output. The Adam moment estimation stochastic optimization approach, e.g., as disclosed in Kingma and Ba, “Adam: A Method for Stochastic Optimization,” ILCR 2015, arXiv.1412.6980v9, 2014, or other optimization algorithm can be used to compute an adaptive learning rate for each of the internal parameters of the model.

It is typical of machine learning models to miss the resonances of datasets with high volatility during prediction. This is because the probability distribution is centered at the off-resonance for each neuron in the output layer, neglecting the local optima. Joint loss optimization functionality in example methods enables collective error correction for the forward prediction task, allowing an accurate prediction at the local optima.

In an experimental training operation, an example training set utilized ˜80% of the 640 collected samples, with the remaining used as a validation set. Each sample was constituted by the full 3×241 LCP absorption, RCP absorption, and CD spectra data points. These 241 data points were generated at 5 nm step intervals within the 400 nm-1600 nm wavelengths. The MSE recorded was 0.000441, which was indicative of the model's accuracy. After training, the validation dataset, which was unseen throughout the training, was used to evaluate the model. Within short prediction intervals, the MDL model exhibited prediction results comparable to simulation data.

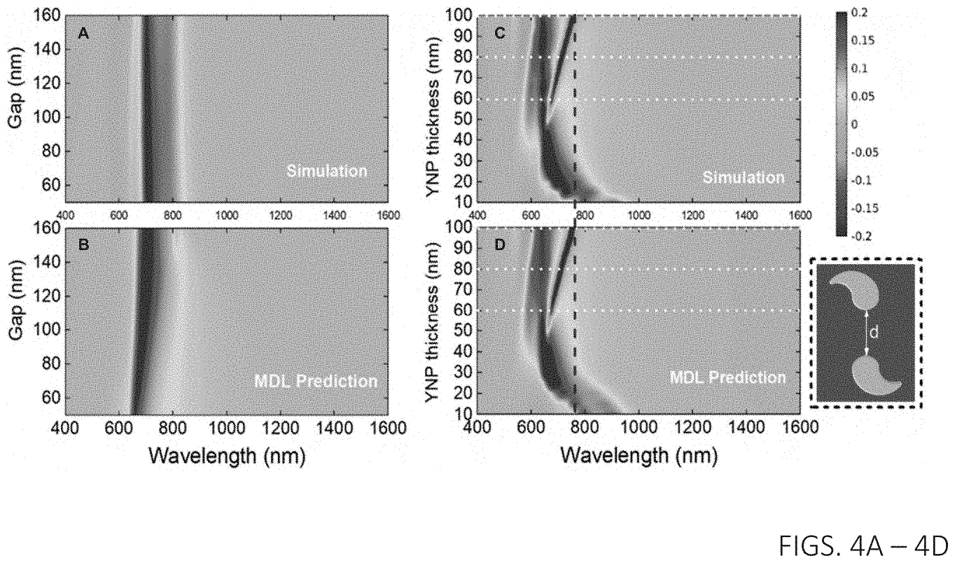

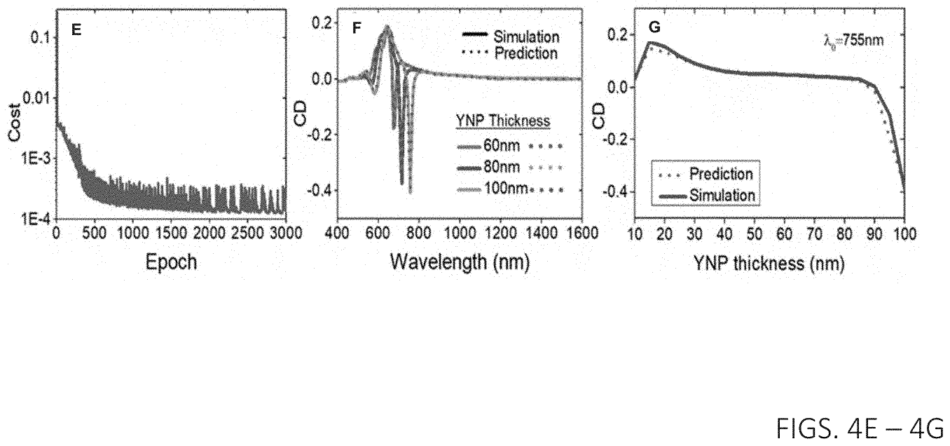

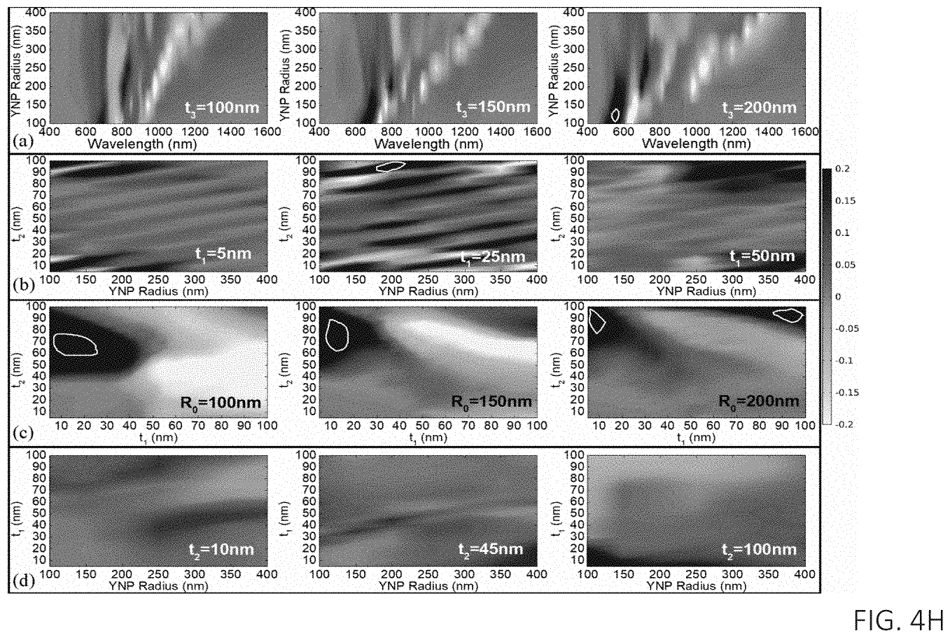

Referring now to FIG. 4, evaluating example MDL model performance, a comparison between numerically simulated (FIG. 4A) and MDL-predicted (FIG. 4B) CD response of an example dimer structure at varying dimer gap distance (length) d (50 nm-160 nm) across the visible and near-IR regime shows a good agreement. Here, R₀=100 nm, t₁=40 nm, t₂=50 nm, and t₃=100 nm. The range of wavelength values corresponding to high and low chiroptical activity are essentially identical.

Numerical simulation (FIG. 4C) and MDL prediction (FIG. 4D) results of the example dimer at varying YNP thickness are also illustrated. The legend has been truncated at ±0.2 for clarity, and the inset illustrates a definition of the gap d. Comparing FIG. 4C (numerical simulation) and FIG. 4D (MDL-prediction) for varying YNP thickness (t₁) shows a good agreement even for ultrathin YNPs. The MDL prediction retains the same wavelength interval as the simulation, providing a full continuous prediction spectrum (241 prediction datapoints). In this case, essential information within short parameter intervals can be retrieved to enrich the analysis, design, and prototyping process.

FIG. 4E shows an example learning curve within 3000 epochs, illustrating the fast convergence of the example MDL model. Further, the single end-to-end MDL model accurately captures both resonant and off-resonant CD signals of the metastructures for varying geometric parameters, as illustrated by FIGS. 4F-4G.

The example training set emphasized larger gap dimensions, which are easier to fabricate consistently, rather than more experimentally challenging dimers with smaller gaps. This bias accounts for small discrepancies observed in FIG. 4B with respect to the simulation values in FIG. 4A, a deviation that can be further reduced by a more homogeneous sampling. The situation is similar, although less pronounced, for the data in FIG. 4D.

Given the design parameter space of the example structure, vast sets of CD responses from varied parameter configurations can be retrieved across the visible and near IR regime (λ₀=400 nm-1600 nm) via the trained MDL model. FIG. 4H is a map plot illustrating MDL-predicted CD progressions, showing the interdependencies among the design parameters of the chiral metamaterial absorber and the evolution of CD generated by the example MDL model with concurrently varying geometric parameter dimensions. For each plot, the unrepresented geometric parameters are held constant during the data generation.

In FIG. 4H, top panel (a) shows evolution of the CD values for varying YNP radius at t₃={100 nm, 150 nm, 200 nm}. A slight shift and broadening of the CD peaks was observed within the high chiroptically active wavelength interval (520 nm-800 nm) for R₀ within the range 100 nm-250 nm at varying t₃. Although the influence of the Au backreflector thickness, t₃, on the CD response is significant, its effect is comparatively small. This is clear when it is considered that the Au layer indeed serves as a reflector, and it should be expected that changes in thickness, when it is already above the penetration depth of the radiation, will not be very impactful to the overall response of the metamaterial.

Second panel (b) compares the CD response by concurrently varying the YNP radius t₂ and polymer thickness at t₁={5 nm, 25 nm, 50 nm}. This plot is obtained at λ₀=780 nm. The effect of the quasi-linear regions was observed, where the CD remains constant irrespective of the coupling gap between the nanoresonators. Therefore, the YNP and PMMA thicknesses together with the YNP radius control the shift in the CD resonances. Varying these sets of parameters yielded highly distinguishable CD map plots as illustrated in panels (b), (c) and (d). Third panel (c) shows CD values at R₀={100 nm, 150 nm, 200 nm} generated by varying concurrently YNP thickness and polymer thickness t₁ and t₂ at λ₀=700 nm, The contour regions correspond to a CD magnitude of 0.5.

Fourth panel (d) illustrates the interaction between the YNP radius and YNP thickness at t₂={10 nm, 45 nm, 100 nm} for λ₀=650 nm. The legend has been truncated at ±0.2 for clarity, but high-CD areas have been highlighted by adding the contour regions corresponding to CD values of 0.5. It can be deduced that the CD magnitude is generally lower for larger values of YNP radius. However, with thinner YNPs (t₁<30 nm), large CD signals can be realized by larger YNP radii (300 nm<R₀<400 nm). For thicker YNPs (t₁>45 nm), large CD signals can be attained for both ultrathin (t₂<20 nm) and thicker (65 nm<t₂<100 nm) PMMAs at larger YNP radius.

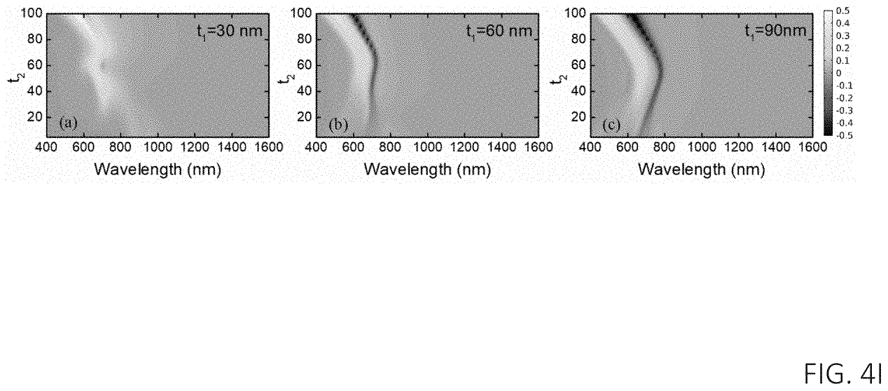

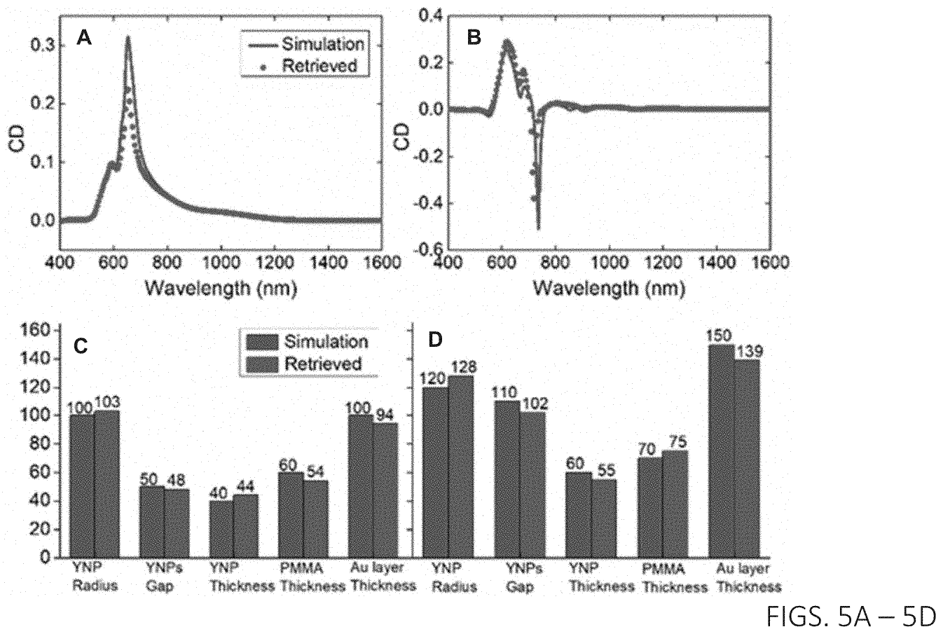

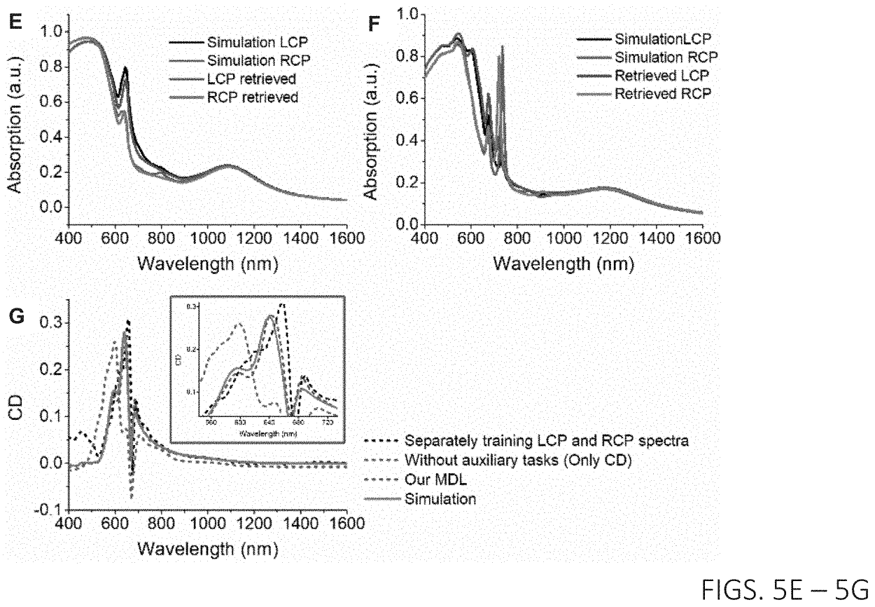

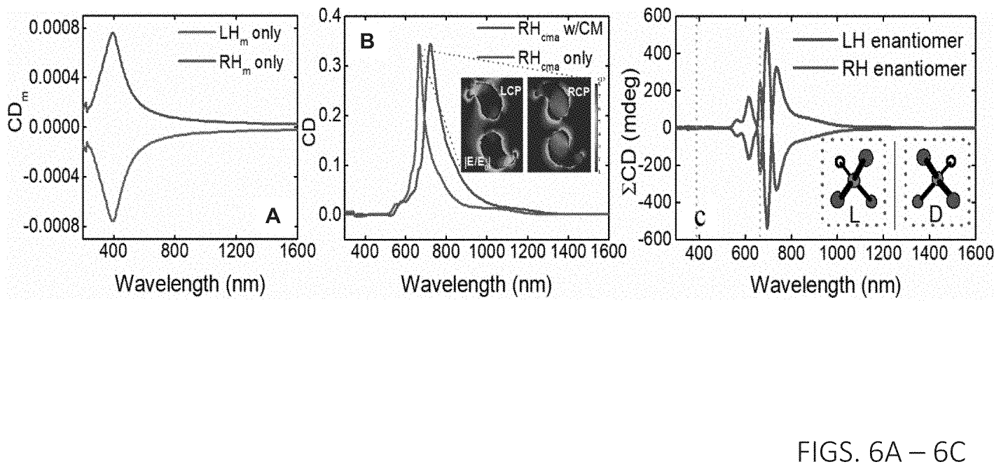

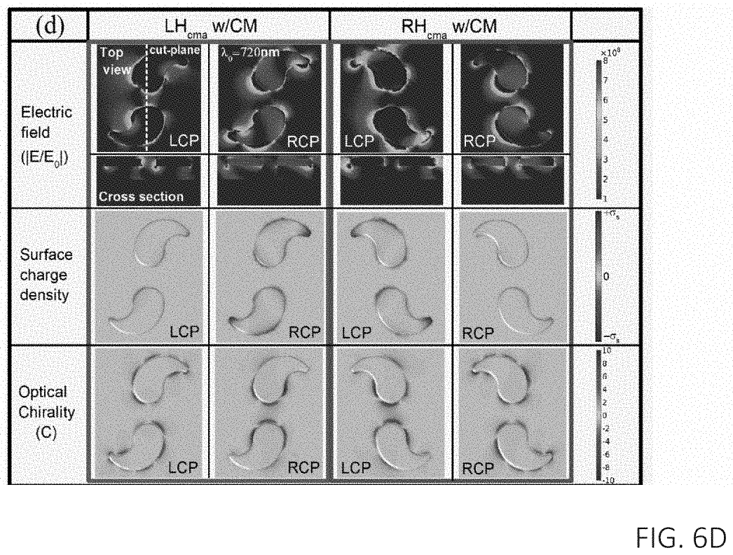

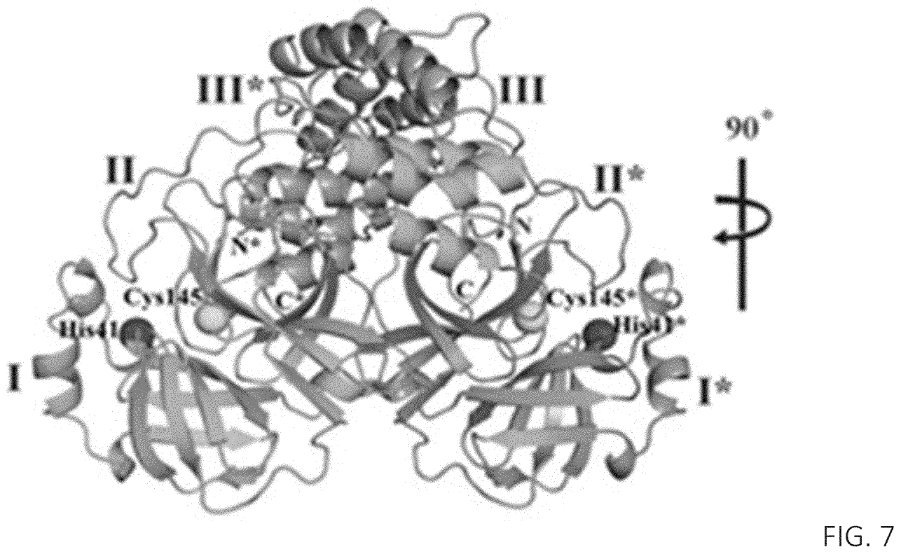

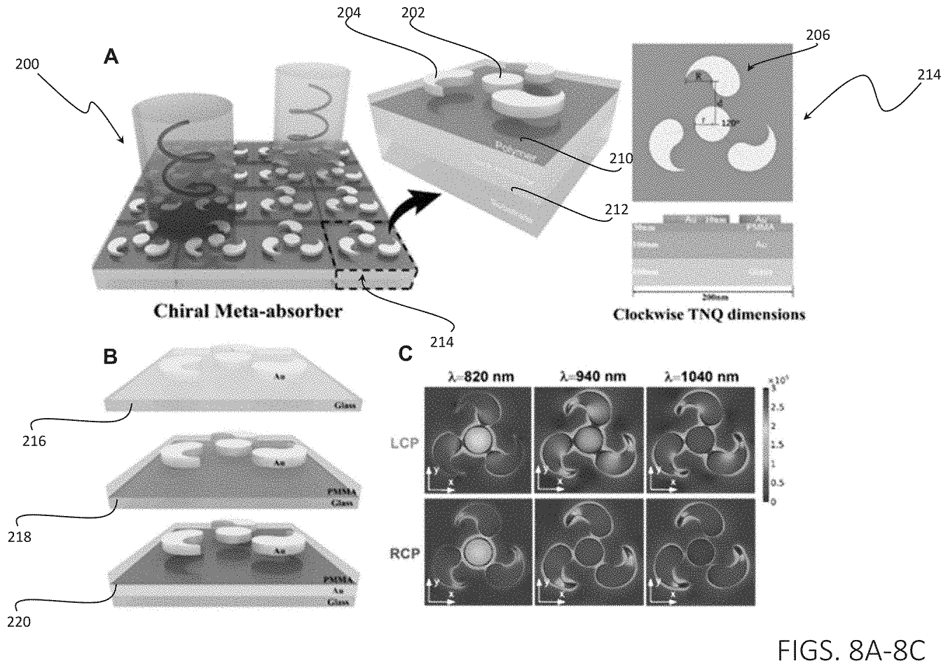

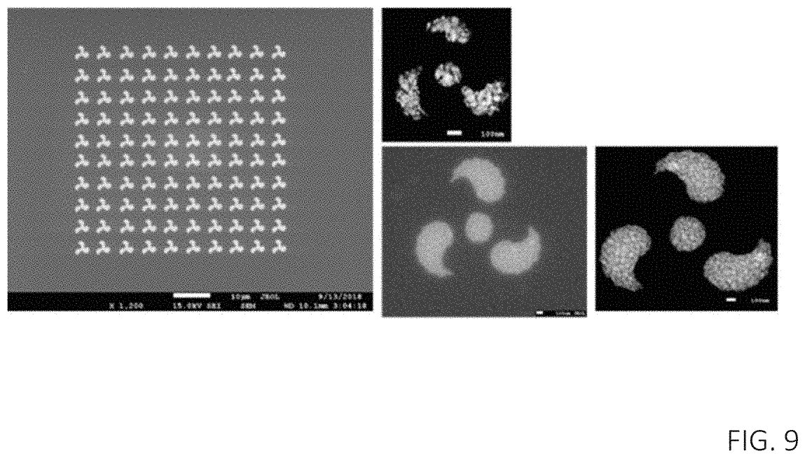

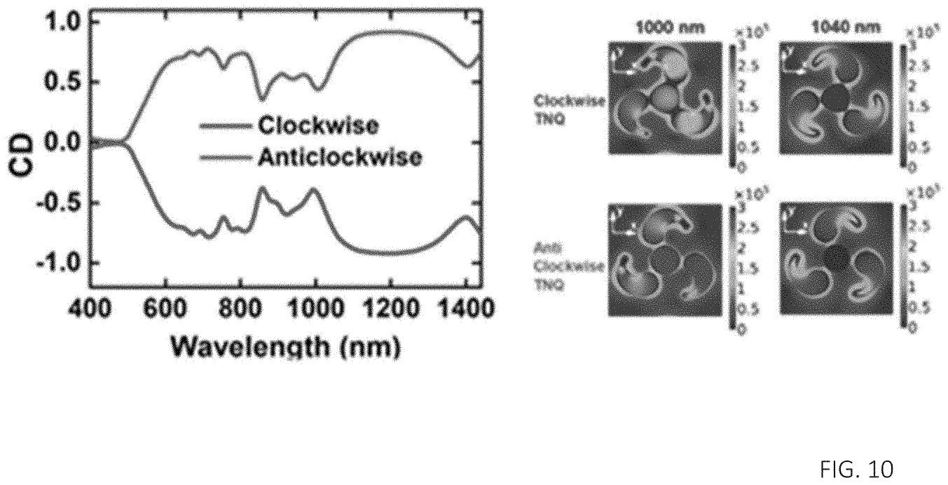

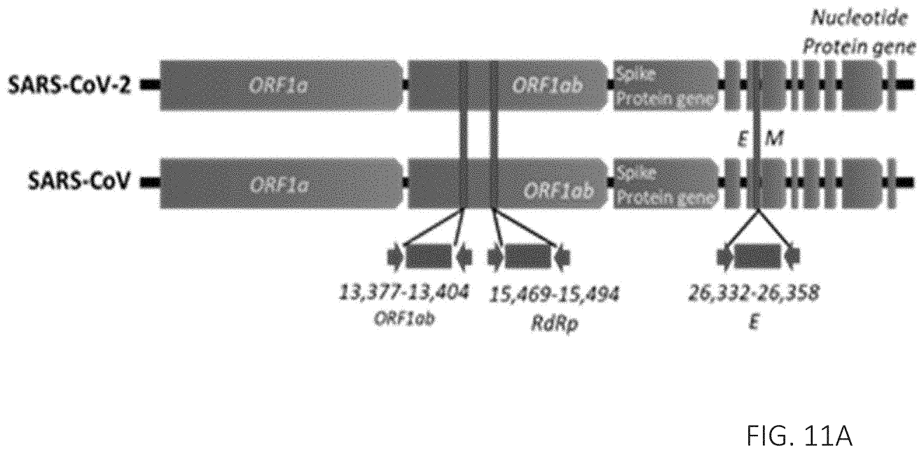

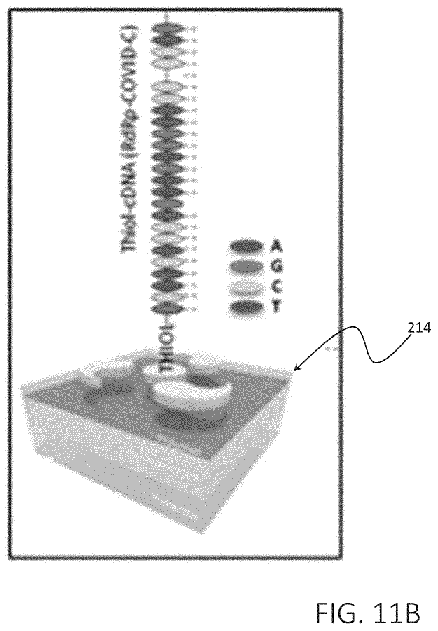

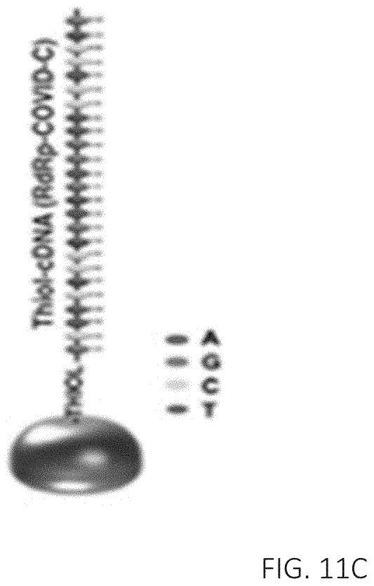

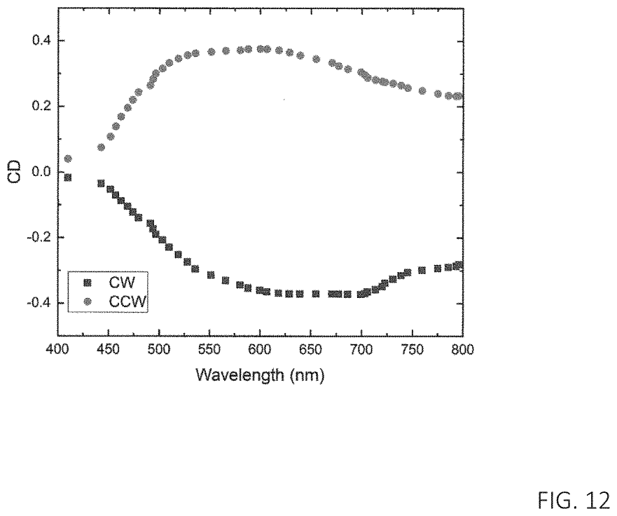

FIG. 41 shows example MDL-predicted CD progressions across the studied wavelengths, including CD evolution, when varying PMMA thickness at t₁={30 nm, 60 nm, 90 nm} for R₀=100 nm. This plot agrees with the above results where one observes only CD maxima for t₁=30 nm but both CD maxima and minima for t₁=60 nm and t₁=90 nm.

The above experiments illustrate capabilities of an example MDL model-based network, and they provide a qualitative understanding of example physical systems. However, it will be appreciated that the output of the example trained neural network can be easily extended to cover many more geometric parameter combinations, and for different wavelengths.

Example MDL approaches can require a much smaller amount of computational resources than producing an equivalently dense dataset through traditional simulation methods. This can be illustrated by comparing both approaches using estimates. For instance, for each combination of geometric parameters, it was found to take a typical processor (e.g., a PC), assumed to consume a total output of its (e.g., 330 W) power source, more than three hours to compute a full spectrum when using COMSOL with precision levels adequate for an example system. This order of magnitude for the duration of the computation is also representative of other simulation packages and frameworks. By contrast, the total energy used for creating an example model (combining the energy required to generate the training set with COMSOL and actually training the network) was approximately 0.63 MWh spanning 80 days using the same example PC. After training, using the example MDL model required less than a second to produce the response for a given geometric parameter set.

On the other hand, for the five example geometric parameters and the example chosen sampling density, the total number of simulations required to obtain the sample result density that the example trained MDL affords would be in the tens of thousands (˜28106 samples) which would require an outrageous number of years (˜10000 years) to produce, with a total energy consumption of approximately 29 GWh using the above example PC. Therefore, in an extreme comparison, an example MDL approach may outperform a naïve simulation-based approach by, for instance, seven orders of magnitude in terms of speed and energy expenditure.

Inverse Design

An inverse design path (IDP) 64 for the example network 60 will now be described. The example inverse design path 64 accounts for the large imbalance between input and output dimensions of the example MDL model 60, namely five geometric parameters compared with the full CD, LCP, and RCP spectra (1×5 versus 3×241 respectively). While it is plausible to apply up- and down-sampling approaches to resolve this imbalance, significant features may be lost in the process, especially in the case of complex structures like the example YNP. Such lost features may introduce prediction errors that compound over a training loop, resulting in wide variations in the retrieved geometric parameters for comparatively small changes in the input spectra.

Reverse-engineering the multitask forward design path 62 can make use of the entire set of CD datapoints and maps each output geometric parameter to the desired full input spectra. The example IDP 64 takes a simulated CD spectrum 102 as input, with the objective of retrieving the geometric parameters required to produce it. To achieve this objective, the example input spectrum can be connected to three isolated dense networks 104 with two layers, responsible for mapping the chiroptical response to a shared latent-space representation 106 with four layers, each composed by 2048 neurons. The shared latent space 106 links to five task-specific layers 108 with each layer managing the output of a designated geometric parameter (FIG. 3).

FIGS. 5A-5D illustrate an inverse design path with an MDL model according to example embodiments, where FIGS. 5A-5B show simulated (green solid lines) and predicted (red dots) CD spectra, and FIGS. 5C-5D show corresponding simulated (green bars) and retrieved (red bars) geometric parameters.

Running this inverter network 64 provides a set of geometric parameters, which will be close to the ones generating the target CD spectrum. Considering the interdependency of these parameters and the high sensitivity of the CD response to them (see, for instance, FIG. 4B), further experiments were conducted to assess the ML-predicted CD spectra and characterize the efficiency of the example inverse retrieval model in obtaining a satisfactory system prediction. For verification, the retrieved geometric parameters were re-fed into the forward prediction path 62, so that a comparison could be drawn between the target CD from the ground-truth geometric parameters and the ML-predicted CD from the ML-retrieved geometric parameters.

FIG. 5A shows a simulated CD serving as a target spectrum (green solid line), with its corresponding geometric parameters in FIG. 5C (green bars). Upon feeding the simulated CD spectrum into the inverse path, the IDP retrieved the geometric parameters in FIG. 5C (red bars). Although it was clear that the inverter network 64 found values that were very close to the ground-truth, the experiments proceeded to verify the retrieval by inputting these geometric parameters into the forward path and comparing its predicted CD spectra response (red dots in FIG. 5A) with the simulated CD spectrum, thus confirming the success of the inverse path.

FIGS. 5B and D were generated following the same procedure. As with FIGS. 5A and C, FIG. 5B shows simulated (green solid lines) and MDL model predicted (red dots) CD spectra, and FIG. 5D shows corresponding simulated (green bars) and retrieved (red bars) geometric parameters. Both input spectra in FIGS. 5A and B were chosen to illustrate the model acting on two profiles with distinct properties (inflection points, negative and positive resonant CD peaks).

Generally, there was a good agreement between simulation and ML-prediction responses, although one could observe slight differences in the spectra that cohere with the small differences between the sets of retrieved and ground-truth geometric parameters. FIGS. 5E-5F shows inverse design plots for simulated and MDL-retrieved absorption spectra comparison for the systems providing the data in (e) FIG. 5A and (f) FIG. 5B.

FIG. 5G compares different approaches to the training of the model such as training with and without the auxiliary tasks as well as separately training the LCP and RCP spectra. For instance, FIG. 5G shows a comparison between the CD values obtained with different variations of the ML techniques: separately training LCP and RCP spectra (black dashes); training only CD without auxiliary tasks (red dashes); training by an example MDL model (blue dashes); and the ground truth (pink solid line). The inset is a zoomed-in image at the resonances. Comparatively better performance in the case of the example MDL model was observed.

MDL-Optimized Dimer Structure in Chiral Biosensing

MDL-optimized chiral plasmonic structures, such as but not limited to the example MDL-optimized dimer structure, can be adopted for various applications, including but not limited to photodetection of circularly polarized light (CPL). For illustrating certain features and benefits, an example use of an MDL-optimized chiral plasmonic structure in sensing adsorbed molecular enantiomers will now be described.

The strength of the chiroptical response from biomolecules is mostly limited by the small magnitude of their geometric chiral features, relative to the periodicity of circularly polarized light (CPL), in comparison with chiral plasmonic nanoantennas. Therefore, it is often necessary to use large molecular concentrations of non-racemic mixtures to provide detectable CD signals. Moreover, chiral biomolecular structures have chiroptical activity in the UV and are thus difficult to detect with common instrumentation. For instance, FIG. 6A shows the weak CD resonant peak of a molecular enantiomer pair in the UV.

The optical activity of chiral molecules can be enhanced by a handedness-preserving Fabry-Perot cavity resonator acting as a metamirror. Such metamirrors selectively reflect one CPL preserving its handedness while absorbing the other. The molecular CD signal can also be enhanced by the interaction with the strong near-field of plasmonic structures, as well as duplicating its CD signal into the visible range, where they are easier to detect.

These advantages, which greatly enhance the detection capabilities of a chiral biomolecular sensor, are in principle independent from the chirality of the plasmonic metamaterial. Non-chiral nanoparticles can offer an effective and efficient sensing of molecular enantiomers by coupling with the chiral molecules. However, local superchiral near fields can offer additional possibilities for molecular CD detection with chiral plasmonic nanostructures on a metal-biomolecular platform. Superchiral fields are those with a larger chirality than CPL, as characterized through their optical chirality, C, and can arise on chiral plasmonic structures illuminated with either linearly or circularly polarized light. Such near fields with enhanced optical chirality cause asymmetric phase differences between the chiral modes in the presence of a chiral dielectric, offering a path for sensing biomolecular chirality.

Example embodiments provide an ML-optimized nano-dimer metastructure in the presence of molecular enantiomers. Theoretical evaluations were conducted to illustrate the behavior of such an ML-optimized nano-dimer metastructure using the excess CD method to quantify the chirality of the molecular sample.

An example metastructure was optimized for value of CD of 0.35. The geometric parameters of the example metamaterial are R₀=100 nm, t₁=30 nm, t₂=50 nm, t₃=200 nm, t₄=200 nm, and d=100 nm. Modelling the chiral molecules, the dimer is covered by a 40 nm thick chiral medium with 1.6 refractive index.

The chiral dielectric medium is modeled following the constitutive equations:

$$\begin{matrix}D = {ɛ}_{0} {ɛ}_{r} E + i ξ B & ( 3 ) \\ H = \frac{B}{{μ}_{0} {μ}_{r}} + i ξ E & ( 4 )\end{matrix}$$

Here, ε₀ and εᵣ are the permittivity of free space and relative permittivity, respectively. Similarly, μ₀ and μᵣ are the permeability of free space and relative permeability, respectively. E and B are the complex electric field and magnetic flux density, respectively. D and H are the electric field displacement and the magnetic field, respectively. ξ is the chirality factor of the molecular sample, which shows very low values for low-density or near-racemic samples.

Using a two-state model for the molecules, ξ can be expressed as a function of frequency as:

$$ξ = {β}_{c} ( \frac{1}{ℏω - {ℏω}_{0} + i {Γ}_{12}} + \frac{1}{ℏω - {ℏω}_{0} + i {Γ}_{12}} ) \qquad ( 5 )$$

where β_{c} controls the magnitude of the chiral asymmetry, ω is the angular frequency of the radiation,

$${ω}_{0} = \frac{2 π {c}_{0}}{{λ}_{mol}}$$

at the molecular excitation wavelength, λₘₒₗ, and Γ₁₂ defines the relaxation rate of the excited molecule, with its indices describing its quantum states. In an example embodiment, λₘₒₗ=380 nm and Γ₁₂=0.41 eV. The expression and values for the chirality factor, ξ, are adopted from A. O. Govorov, Z. Fan, P. Hernandez, J. M. Slocik, and R. R. Naik, “Theory of circular dichroism of nanomaterials comprising chiral molecules and nanocrystals: Plasmon enhancement, dipole interactions, and dielectric effects,” Nano Lett. 10, 1374-1382 (2010) and R. Tullius, G. W. Platt, L. Khosravi Khorashad, N. Gadegaard, A. J. Lapthorn, V. M. Rotello, G. Cooke, L. D. Barron, A. O. Govorov, A. S. Karimullah, and M. Kadodwala, “Superchiral Plasmonic Phase Sensitivity for Fingerprinting of Protein Interface Structure,” ACS Nano 11, 12049-12056 (2017), and follow from the quantum equation of motion for the electronic density matrix when assuming a dilute molecular sample.

The excess CD method was used to compute the chiral properties of the molecules, when in interaction with left-handed and right-handed chiral metamaterial (LH_{cma} and RH_{cma} respectively). Results of enantiomer detection are shown in FIGS. 6A-6D. FIG. 6A shows the CD spectra of (red) left-handed medium (LHₘ), and (blue) right-handed medium (RHₘ) with molecular CD resonance (λₘ=380 nm) in the UV. FIG. 6B shows a CD spectra comparison of the right-handed chiral metamaterial absorber (RH_{cma}) with (blue) and without (red) chiral medium (CM). The inset of FIG. 6B shows the electric field at the plasmonic resonance, λₚ, (λₚ=665 nm) of the bare chiral metamaterial absorber for LCP and RCP light. FIG. 6C shows a CD summation to remove metamaterial background CD signal to reveal the LH (blue solid line) and RH (green solid line) enantiomer pair CD signals, where λₘ, λₚ, and λₘₚ represent the resonant wavelengths for the CD of the bare molecules, the plasmonic chiral metamaterial absorber, and the metamaterial covered with chiral media, respectively. The inset of FIG. 6C is a schematic representation of an enantiomeric protein molecular pair (L and D isomers). FIG. 6D shows electric field, surface charge density, and optical chirality density distributions of the example chiral metamaterial absorber covered by chiral media (CM) at λₘₚ=720 nm.

In example experiments, a baseline CD was calculated from the metamaterial absorber with racemate (ξ=0) molecular coverage. In the process, a distinguishable dielectric medium-induced CD redshift (˜55 nm) was observed, as illustrated in FIG. 6B. This redshift arises from the refractive index of the molecular coverage, which is larger than that of the surrounding medium (air). The inset shows the local field enhancement of the bare metamaterial absorber without biomolecules, illustrating its strong and chiral near-field.

Then, one can compute the sum of the CD signal of the two YNP metamaterial absorbers, with chiral and racemate molecular coverage, yielding a change in CD response associated with the biomolecules of opposite handedness. The coupling of the enhanced plasmonic near-field to the molecules is critical for biomolecular sensing. FIG. 6C illustrates how the CD signal of the chiral molecules shifts its peak to the visible range and increases its magnitude by approximately a factor of two. The excess CD signal over that of the chiral metamaterial absorber, produced by the coverage of molecular enantiomers, is a detectable magnitude that allows one to characterize the presence and handedness of chiral biomolecules, as illustrated in FIG. 6C.

Additional example features of chiral properties of the example metastructure include its near-field enhancement, surface charge density, and optical chirality parameter C. The latter is calculated, for harmonically oscillating fields, as:

$$C = - \frac{{ɛ}_{0}}{2} ω Im ( {E}^{*} \cdot B ) \qquad ( 6 )$$

FIG. 6D compares these three variables for the LH_{cma} and RH_{cma} systems, under the dielectric chiral medium described above. The first row is the top and cross-sectional view of the electric field enhancement distribution of the LH_{cma} (red border) and RH_{cma} (green border), when illuminated by LCP and RCP light. A yz cross-sectional plane is taken to show the field enhancement inside the layered metamaterial. The surface charge density is illustrated in the second row, and the optical chirality maps in the third row. The electric field, surface charge density and optical chirality are evaluated at the chiral media-modified plasmonic resonance, λₘₚ=720 nm. Significantly, the optical chirality maps reveal the superchiral near field regions arising from the curvature and chiral geometry of the nanoantennas.

Protein Analysis and Virus Detection Using Chiral Metamaterial Structures

Example embodiments employ MDL-based models to provide metamaterial structures for protein analysis and separation. Some example embodiments can be used for diagnostic applications. One nonlimiting example application is detection of a coronavirus.

COVID-19 has resulted in many deaths, and it has significantly changed the human lifestyle for the foreseeable future. Rapid and reliable diagnosis of viruses such as but not limited to SARS-CoV-2 is an essential need for control of this or other epidemics.

Several molecular diagnostic techniques have been identified for the detection of SARS-CoV-2 Virus. One of the most sensitive techniques for the detection of any virus is reverse transcription polymerase chain reaction (RT-PCR) based assay. COVID-19 can be detected by the identification of the SARS-CoV-2 RNA. One prevalent diagnostic technique in the United States involves the RNA-dependent RNA polymerase (RdRp) sequence for RT-PCR molecular assays. The SARS-CoV-2 RNA is generally detectable in nasal, nasopharyngeal, and oropharyngeal swabs. This test qualitatively detects the SARS-CoV-2 using a dual target assay for RdRp and N-genes. The qualitative detection of nucleic acid from the SARS-CoV-2 is possible by rapidly making many copies of a particular sequence. The viral load is estimated from the photoluminescence intensity of the molecules that are released into the buffer solution in real time as the duplicate copies are created by the amplification enzymes.

However, RT-PCR based tests can fail due to the amplification of spurious nucleic acid contaminations. The RT-PCR tests for SARS-CoV-2 detection have yielded significant false negative tests specifically for samples from the upper respiratory tract. Recent reports have shown that the false positive results from the nasal swabs can be as high as 48% and accordingly the test can be very unreliable.

An alternate mode of diagnostics is necessary to complement the RT-PCR based assay. However, approaches based on cell culture or diagnostics CT scan are time-consuming and not very appropriate for real-time analysis.

Light-based biosensing techniques may be considered as a reliable modality for real-time detection and as an alternative to biochemical or molecular assays. Localized surface plasmon (LSP)-based biosensing systems can rapidly detect and quantify virus interaction with the metal surface, as localized plasmon interactions are strongly modified by any local variation of the refractive index or surface potential due to molecular binding.

Recently, resonant surface plasmon interaction has been utilized to study the structure of SARS CoV-2 spike protein. Real-time and label-free detection of SARS CoV-2 using surface plasmon sensors has been disclosed. This sensing technique is based on two different plasmonic interactions, one due to localized surface plasmon sensing transduction, and a plasmonic photothermal interaction induced by light at two different wavelengths and incident angles. This sensing technique demonstrates the feasibility of using RNA based hybridization on Au nano-islands as an effective means for the development of on-chip plasmonic sensors. This technique is based on attenuated total-reflection technique for exciting the localized plasmon modes. This work also demonstrated the stability and specificity of binding the desired oligonucleotide sequence to the active plasmonic platform for developing an optical sensor that can overcome the interference in the output signal of an actual binding event from binding of multiple nonspecific sequences.

However, to increase the sensitivity of the sensor, the above approach utilized the Fourier transformed phase from the ATR spectral interferograms of the local plasmon response. The dual excitation process and the angle dependent sensing process makes the technique rather indirect and complex.

Some example embodiments herein provide a COVID-19 sensing platform based on the chiral optical response of nucleic acids with specific hybridization of complementary bases and proteins with predictable homochirality or optical isomerism. According to some embodiments, plasmonic meta-surface structures are improved for the detection of SARS-CoV-2 using a machine learning (ML) algorithm. Example MDL models can be used to optimize or improve and develop cDNA based chiral plasmonic sensors for the detection of SARS-CoV-2.

Example experiments investigated the stability of SARS-CoV-2 protomers on Au plasmonic meta-structures for sensing, and assess the feasibility of using rotational anisotropic second harmonic generation (RA-SHG) for SARS-CoV-2 detection.

A real time COVID-19 sensor according to some embodiments is based on a principle that the homochirality of the cDNA molecule and protomers in SARS CoV-2 can be utilized to induce a change in their interaction with circularly polarized light or a chiral photon. This linear and nonlinear interaction can be enhanced in example embodiments to develop a reliable biosensing platform using chiral plasmonic meta-surfaces, which can be optimized using machine learning tools.

Circular dichroism (CD) spectroscopy is a complementary technique to X-ray crystallography, cryoelectron microscopy, and NMR spectroscopy. It provides a lucrative avenue to studying chiral biomolecules such as amino acids peptides, RNA, and DNA. Most naturally occurring molecules are inherently chiral such that their interaction with circularly polarized light generates significantly different chiroptical response for left circularly polarized (LCP) and right circularly polarized (RCP) lights. This allows for a recognition function for discriminating and sensing biomolecules based on their unique chiroptical response. Although many essential molecules characterizing a virus or bacteria can exist in two mirror-image forms, referred to as “left-handed” and “right-handed”, proteins are exclusively composed of left-handed amino acids, while RNA in the RNA-virus contain only right-handed sugars.

FIG. 7 illustrates the three-dimensional structure of SARS-CoV-2. The two dimer's protomers are shown in light blue and orange. Amino acid residues of the catalytic site are indicated as yellow spheres for Cys145 and blue spheres for His41. The residues from protomer B in orange is represented by an asterisk (*). Black spheres indicate the positions of Ala285 for each of the two domains III. Chain termini are labeled N and C for molecule A (light blue) and N* and C* for molecule B (orange).

The structure of the proteins or RNA of SARS-COV-2 virus shows a homochirality. Domain I of the structure shows structural symmetry around the central axis including the amino acid residues of the catalytic sites Cys145 and His41. The various proteins constituting the virus can be homochiral in nature. The two enantiomers of the protomers or RNA in SARS-COV-2 and the spheroid virus in its entirety will interact differently with circularly polarized light or chiral photons due to their right- or left-handedness, which forms the basis of example optical techniques for chiral molecular sensing.

Thus, SARS-COV-2 virus can be detected by the change in the differential optical absorption of left and right circularly polarized light. The RNA or protomers of SARS-COV-2 will preferentially absorb or reflect one direction of circularly polarized light, which can be measured by CD spectroscopy. The CD depends on the chirality of the enantiomers and can also be enhanced by the chirality of the light source.

However, the strength of the chiroptical response from nucleic acids and proteins can be hindered by the small asymmetry of their molecular anisotropic feature compared to wavelength of the light, especially when the molecular concentration is low. This low sensitivity can result in a bottleneck for early detection. This can be overcome by using plasmonic nanostructures that induce a near-field exciton plasmon coupling due to the enhanced electromagnetic field under resonant excitation. Surface plasmon interaction has enhanced the circular dichroism of DNA linked Au nanorods. However, most of the protomers or DNA/RNA have molecular absorption in the ultraviolet (UV) region that makes detection challenging without UV optics.

Example plasmonic meta-materials and meta-structures provided herein can not only enhance the strength of the CD but can also red-shift this strong interaction of chiral molecules with the plasmonic near-field to the longer wavelength regime. Various chiral geometries have demonstrated huge CD signals, including varying configurations of gammadions, helices, spirals, etc.

Planar plasmonic nanostructures are relatively convenient to fabricate and characterize using example methods. Additionally, in gapped cluster of plasmonic nanostructures such as nanodimers, nanotetramers, and nanoheptamers, interparticle plasmonic resonant coupling can further amplify the generated CD response owing to multiple plasmon modes, i.e., the dipolar and quadrupolar modes. In such structures, aside from varying the parameters of the assembly, it is possible to tune the chiro-optical response using inter- and intra-plasmon coupling. The strongly coupled structures thus translate into strong CD.

Example meta-absorbers offer various benefits over conventional absorbers including but not limited to wider adaptability, effectiveness, and miniaturization. Example chiral plasmonic metamaterials exhibit inherent CD signals whose amplitude depends on the degree of chiral asymmetry of their geometry and thus present an avenue for designing and manipulating nanoscale chiroptical effects. The magnitude of the CD or the figure of merit of the device can be improved by the chirality of the light source.

In an example method, using a multitask deep-learning (MDL) architecture composed of a single end-end bidirectional architecture such as the network 60 shown in FIG. 3, a three-dimensional triskelion nanostructure based plasmonic metamaterial superchiral structure is provided to achieve strong optical chirality and detect small changes in the chiral interaction. The optimization can be carried out using inverse retrieval operations for learning of the primary task (circular dichroism (CD)), and can be aided by two auxiliary tasks, namely the wavelength of operation and input material properties (such as layer thickness, dielectric function etc.). An example MDL model uses FEM simulations or experimental data as training sets for the optimization.

Similar to equation (5) above, according to some embodiments, the chirality factor ξ of the molecular sample can be estimated from:

$$ξ = {β}_{c} ( \frac{1}{ℏω + {ℏω}_{0} + i {Γ}_{12}} + \frac{1}{ℏω - {ℏω}_{0} + i {Γ}_{12}} ) \qquad ( 7 )$$

where β_{c} controls the magnitude of the chiral asymmetry, w is the angular frequency of the radiation,

$${ω}_{0} = \frac{2 π {c}_{0}}{{λ}_{mol}}$$

at the molecular excitation wavelength, λₘₒₗ, and Γ₁₂ defines the relaxation rate of the excited molecule, with its indices describing its quantum states.

The chiroptical properties of any chiral structures are characterized by the CD and g-factor. These properties reflect inherent topological properties of the chiral matter at the nano/micro scale. According to some embodiments, simulations and experiments are based on the CD in the absorption domain.

Absorption is calculated as

A_{LCP(RCP)}=1−R_{LCP(RCP)}−T_{LCP(RCP)} (8)

Where, R_{LCP(RCP)} and T_{LCP(RCP)} are the reflectance and transmittance for LCP and RCP incidence, respectively.

The CD and g-factor in the absorption are estimated from

$$CD = {A}_{LCP} - {A}_{RCP} , {g}_{CD} = \frac{{A}_{LCP} - {A}_{RCP}}{\frac{[ {A}_{LCP} + {A}_{RCP} ]}{2}} \qquad ( 9 )$$

Where, A_{LCP} and A_{RCP} are respectively the absorbances of the chiral nanostructure illuminated by left and right circularly polarized beams.

The example multifunctional optical sensor has characteristics of conventional plasmonic sensors, as the specific binding of any analyte to the metal-nanostructures can result in a shift of the absorption wavelength of the plasmonic response. Additionally, the response to the difference between right-handed and left-handed polarized light to a homo-chiral molecule bound to the superchiral plasmonic meta-surface can also yield information about a binding event.

Referring again to FIGS. 6A-6D, considering a chiral molecule with absorption at λₘₒₗ, =380 nm, FIG. 6A shows an example CD response with respect to left- and right-handed circularly polarized light. FIG. 6B shows the shift in the optical spectrum of the plasomonic meta-surface due to the binding of the chiral molecule. The baseline CD of the plasmonic metamaterial absorber calculated with racemate (ξ=0) molecular coverage shows the resonant plasmon peak at the resonance.

The presence of a molecule redshifts the CD spectrum by ˜55 nm due to the modification of the dielectric response of the medium. The magnitude of the CD also increases by a factor of two due to coverage of molecular enantiomers over the chiral metamaterial absorber (FIG. 6C). The presence of a chiral molecule can be also detected from the estimation of the effective CD of the isomer or the homochiral molecule from its interaction with the right and left circularly polarized light. The enhanced signal allows characterization the presence of biomolecules due to its interaction with chiral photons from the metamaterial absorber.

It is also possible to resonantly excite the plasmonic metastructures to induce local heating and effect in situ hybridization for a real-time and label-free detection of viral sequences, as provided in more detail herein.

Design and Fabrication of Triskelion Superchiral Plasmonic Metamaterial Sensor

Referring to FIG. 8, in an example embodiment, an example detection device (sensor) 200 includes a top asymmetric Au metastructure constituted by a central nanodisk 202 with encompassing three “yin-yang-shaped” split-ring nanoantennas 204 in clockwise direction to provide triskelion structures (TNQs) 206. The central nanodisk 202 and flanking nanoantenna radii 204 are optimized using the example MDL machine learning network shown in FIG. 3. The initial separation gap, d, between the nanodisk 202 and nanoantennas 204 from their centers (right top portion of FIG. 8A) is optimized to induce maximum interparticle coupling.

The optical response of the sensor 200 can be modified based on, for instance, the limitations of the device fabrication process. For instance, it may be challenging to develop small (e.g., sub-100 nm) nanostructures on a non-conducting transparent substrate using electron-beam lithography.

These triskelion structures (TNQ) 206 were fabricated on a 30 nm thin polymethyl methacrylate (PMMA) polymer layer 210 that was deposited on a gold coated quartz 212 as the back-reflecting surface. Such a structural combination can yield optical absorption properties approaching perfect absorption (absorption close to unity).

In an example operation of the sensor 200, circularly polarized light (CPL) is irradiated from the top boundaries and the reflected and transmitted lights are collected at the top and bottom boundaries. Due to the effect of the Au back-reflector, there is an enhanced differential absorption between LCP and RCP light. There is also an inversion in the CD response of the metamaterial absorber for YNP enantiomer pairs.

FIG. 8A shows the metastructure 200 including an example chiral meta-absorber array with definition of incident circularly polarized lights and a unit cell 214 with its dimensions. FIG. 8B shows three example metastructure configurations: TNQ/Glass 216, TNQ/PMMA/Glass 218, and TNQ/PMMA/Au/Glass 220.

FIG. 8C shows a comparison of the surface electric field distribution maps for the TNQ/Glass metastructure 216 at selected wavelengths upon LCP (first row) and RCP (second row) incidence revealing differences in field patterns. In FIG. 8C the field distribution is viewed on the xy-plane at z=0 (the TNQ-PMMA interface). The nanodisk radius r and gap between nanodisk and surrounding nanoantennas d, are 25 nm and 50 nm respectively.

In experiments, example chiral structures were fabricated as a plasmonic meta-surface on a transparent substrate using electron beam evaporation and electron beam lithography processes. A transparent silicon dioxide/quartz substrate was prepared with an appropriate combination of Au/dielectric layers. Example structures were composed of SiO₂ (quartz) coated with 2 nm of chromium for the adhesion of the reflective metallic mirror layer of 100 nm on the substrate. The Au layer was covered with 30 nm Alumina (Al₂O₃), which has a slightly higher refractive index. The higher index of the Al₂O₃ of the spacer used for the convenience of fabrication provides more dielectric confinement in addition to the confinement due to the organic PMMA layer on its surface as estimated in the simulations. These metal and dielectric layers were deposited using electron beam evaporation.

To provide the photoresist, polymethyl methacrylate (PMMA) was spin coated at 4000 rpm for 30 seconds and baked at 150° C. for 90 seconds. The triskelion structure was written on the substrate using a conventional electron beam lithography (EBL) technique. To enhance the adhesion of the Au films to the poly(methyl methacrylate) (PMMA) substrates, an ultraviolet (UV)-ozone surface modification was performed. The exposure time and curing conditions can be optimized to ensure that the dielectric constant of the UV-ozone modified is not significantly modified due to the UV exposure. The electric field was maintained at 15 KV, with a current of 240 pA, with exposure of 200 μC/cm² dose. After development of the exposed resist, the 30 nm Au layer was deposited by electron beam evaporation.

FIG. 9 illustrates an example nanofabricated chiral plasmonic meta-surface structure. These structures can be fabricated on glass substrates using nanoimprint technology, such as for batch production of an optical sensing device structure (e.g., for detecting SARS COV-2). Various dimensions of the meta-surfaces were fabricated as shown in FIG. 9. Low energy electron beam can be used to optimize the fabrication process, e.g., for development of the sharp-tips of the yin-yang structure for very small (e.g., sub-100 nm) structures.

The physically fabricated device structures were used as input parameters in the machine learning code for the estimation of the optical response shown in FIG. 8. A smaller dimension of the plasmonic nanostructures constituting the meta-surface or Ag metal is more useful for the operation of the device in the shorter wavelength. The prototype device was tested using Au plasmonic meta-surface which has an optical response in the longer wavelength region.

The example MDL was used for studying the effect of changes in the chiral response of the TNQ for two different orientations of the clockwise and anticlockwise flanking split ring nano-antennas. FIG. 10 shows a large response of the chiral optical anisotropy induced by example triskelion plasmonic structures. A large difference in the circular dichroism induced by the orientation of the chiral triskelion structures is apparent. It also can be seen that the light from a clockwise triskelion structure and an anticlockwise triskelion structure have a difference in the field distribution pattern at different wavelengths.

The use of a right- and left-handed polarized light can yield a significant wavelength dependent response. Moreover, the binding of a L or D isomer or a homochiral DNA (right-handed) or protein (left-handed) to these TNQ structures will affect this field distribution differently. Depending on the right-handed or left-handed molecule, clockwise or anticlockwise triskelion structure can be selected and tested.

cDNA and Protein-Based SARS CoV-2 Sensors

In alternative embodiments, provided are devices acting as sensors for detecting a nucleotide such as a cDNA and/or a protein such as a protein from a pathogen such as a bacteria or a virus, wherein the cDNA or protein can be derived from a Coronavirus, for example, a SARS CoV-2 or COVID-19 virus.

In alternative embodiments, devices acting as sensors as provided herein can also be used to detect any protein for diagnostic or therapeutic purposes. For example, the protein can be derived from a blood or plasma sample, or a tissue sample, for example, a biopsy, from an individual in need thereof, for example, a human or an animal.

In alternative embodiments, any protein or nucleic acid can be attached to or disposed as a “nanoantenna” of a device as provided for the purposes of detecting any biological molecule, including any protein, nucleic acid (such as a DNA, cDNA or RNA), lipid and/or polysaccharide.

Coronaviruses are enveloped positive sense (plus-strand) RNA viruses. The CoV particles have a spherical shape with a diameter of roughly 100 nm. The central pad 202 of an example triskelion structure (TNQ) 206 is comparable to the size of these particles. CoV genomes range between 27 and 32 kb, representing the largest RNA genome known to date. It is therefore very efficient to detect the optical response of the RNA for the identification of SARS COV-19 virus.

The virion volume of CoV viruses is 9.0×105 nm³, which is one of the highest in any single strand positive sense RNA virus and makes it very deadly. Acquisition and isolation of SARS-CoV-2 samples from infected patients is difficult. Handling of these live viruses requires a biosafety level-3 and/or biosafety level-4 laboratory. Therefore, the World Health Organization (WHO) has suggested the use of two closely related oligonucleotide sequences from SARS-CoV-2 for the development of diagnostic sensors as shown in FIG. 7.

According to example embodiments, the development of a linear optical biosensor can be based on using the protein related to SARS-CoV-2. The angiotensin-converting enzyme 2 (ACE2) is a transmembrane protein that serves as the main entry point into cells for SARS-COV-2 virus. The angiotensin-converting enzyme 2 (ACE2) binds to SARS-COV-2 more strongly than SARS-COV. Thus, the kinetics of this binding can be detected by surface plasmon interaction.

The binding of SARS-CoV-2 protein to Au based surface plasmons has been previously disclosed. The binding is expected to modify the chiral response of ACE2. The ACE2 protein has an intracellular C-terminal collectrin renal amino acid transporter domain and an outside N-terminal peptidase M2 domain.

Artificially synthesized oligonucleotides RdRb-COVID-C and the ORF1abCOVID-C with RdRp-SARS-C for a comparative analysis can be procured (e.g., from commercial vendors or CDC recommended sources) and used for the development of example biosensors. These particular sequences can be attached to the gold plasmonic chiral nanostructures using thiol chemistry as shown by example in FIG. 11(b) (showing unit cell 214). The thiol cDNA receptors to bind the oligonucleotides on the gold nano-antennae and the meta-surface structures can be procured from similar sources.

The (HS−) group attached at the head of (5′-) of each strand of RdRp-COVID-C, RdRp-SARS-C and ORF1ab-COVID-C forms the complementary sequence to bind to the Au nanostructures. The targeted sequences for functionalization of the Au triskelion (TNQ) include:

(SEQ ID NO: 1)
RdRp-COVID-C
-Thiol-5′-GCATC TCCTG ATGAG GTTCC ACCTG-3′
(SEQ ID NO: 2)
RdRp-SARS-C
-Thiol-5′-GCATC ACCGG ATGAT GTTCC ACCTG G-3′
(SEQ ID NO: 3)
ORF1ab-COVID-C
-Thiol-5′-CCA TAACC TTTCC ACATA CCGCA GACGG-3′

The large viral volume is expected to significantly alter the chiral-response of the plasmonic TNQ structure for a very effective sensing modality. With specific binding of the membrane protein to the Au nanostructures, the sensing efficiency is expected to be even more pronounced as the plasmonic meta-structure is constituted of several TNQ structures.

Example sensors were tested using synthetic oligonucleotides identified above for proof of concept. The absorption data of a synthetic oligomer attached to the gold triskelion meta-surfaces on a quartz substrate showed a large difference at the plasmon frequency for the left-handed polarized light (CVV) compared to the right-handed polarized light (CCVV) (FIG. 12).

The chiral response of the ACE2 protein bound to plasmonic meta-structures according to example embodiments was studied. This amino terminated domain can be immobilized on the Au metal chiral structures using standard conjugation chemistry. HS-PEG-COOH and HS—(CH2)11-EG6-OH (molar ratio, 1:10) at a total concentration of 1 mM can be printed on a fabricated chiro-plasmonic meta-surface as shown in FIG. 9. In case of protein binding, the surface plasmons pads is incubated to allow the formation of a uniform self-assembled mono-layer. The stability of the binding event can be optimized, and the chiral response of the ACE2 protein bound to the plasmonic meta-surface can be assessed. The binding event is expected to also modify the plasmon frequency differently than the cDNA hybridization.

Select sequences of the SARS-COV-2 virus can be used for the selective detection. The sequences can be attached to the gold plasmonic chiral nanostructures using thiol chemistry as shown in FIG. 11A.

Protein Functionalization

The angiotensin-converting enzyme 2 (ACE2) is a transmembrane protein that serves as the main entry point into cells for SARS-COV-19 virus. The angiotensin-converting enzyme 2 (ACE2) binds to SARS-COV-19 more strongly than SARS-COV. Thus, the kinetics of this binding can be detected by surface plasmon interaction. The ACE2 protein contains a C-terminal collectrin renal amino acid transporter domain and an N-termonal peptidase M2 domain [*]. This amino terminated domain can be immobilized on the Au metal chiral structures using standard conjugation chemistry.

HS-PEG-COOH and HS—(CH2)11-EG6-OH (molar ratio, 1:10) at a total concentration of 1 mM can be printed on the chiral-sensor chip shown in FIG. 9. Chips can be incubated to allow the formation of a uniform self-assembled mono-layer.

Referring again to FIG. 10, an example machine learning (ML) aided algorithm used for the study of chirality of the cDNA molecule shows a large response of the chiral optical anisotropy induced by example TNQ plasmonic structures. FIG. 10 (left) illustrates CD spectra for clockwise (purple) and anticlockwise (pink) flanking split ring nanoantennas. FIG. 10 (right) illustrates the spatial field distribution comparison for clockwise and anticlockwise Au TNQ at selected wavelengths (λ=1000 nm and 1400 nm).

FIG. 10 illustrates a large difference in the circular dichroism induced by chiral triskelion structures. It is observed that molecules attached to a clockwise triskelion structure and an anticlockwise triskelion structure have a difference in the large circular dichroism. This indicates that the use of right- and left-handed polarized light can yield a significant wavelength dependent response.

Referring to FIG. 12, sensors according to example embodiments were tested using synthetic oligonucleotides. RdRp-COVID, RdRp-SARS, ORF1abCOVID, E sequence, and their thiol-cDNA receptors, including the RdRp-COVID-C, RdRp-SARS-C, and ORF1ab-COVID-C, E-C, were synthesized and provided by Microsynth (Balgach, Switzerland). The absorption data of the thiol-cDNA oligomer attached to the gold triskelion meta-surfaces showed a large difference at the plasmon frequency for the left-handed polarized light and almost no interaction of the light for the right-handed polarized light.

Use of Rotational Anisotropic Second Harmonic Generation (RA-SHG) for SARS-CoV-2 Detection

Rotational Anisotropy Second Harmonic Generation (RA-SHG) microscopy has the twin advantages of increased spatial resolution and sensitivity to molecular symmetry. As the image is detected at the second harmonics rather than the fundamental wavelength of the excitation laser, the optical resolution is beyond the diffraction limit of the fundamental wavelength, approaching 100 nm or smaller.

The COV particles being 100 nm spherical particles, an example imaging tool according to some embodiments can be used to optically probe the properties of a single COV particle conjugated to a metal nanoparticle. Spatial resolution of less than over an order of the wavelength of light (λ/10) can be achieved without near-field optics. The sensitivity to symmetry can be realized by polarizing the excitation laser. The second harmonic generated (SHG) intensity is highly sensitive to the orientation of the sample relative to the polarization of the laser. Previous disclosures using single-point measurement techniques have exploited this property by rotating the sample.

According to some embodiments, a Rotational Anisotropic Second Harmonic Generation (RA-HG) microscope system uses a galvo-mirror scanning system to raster the laser across the sample and includes telecentric optics to correct for angular deviation at the image plane. A Glan-Taylor polarizer coupled with a half-wave plate can be used to both clean up the polarization state of the beam and provide control over the excitation power at the sample plane. An additional half-wave plate allows rotation of the input polarization field relative to the sample, while another half-wave plate and an analyzer between the collection objective and the detector allows selection of the component of the SHG, THG, or higher harmonics for collection, (parallel or perpendicular). The example detector is a back-illuminated sCMOS camera, with a nominal quantum efficiency of 95% in the region of the spectrum used above.

The foregoing description is merely illustrative in nature and is in no way intended to limit the disclosure, its application, or uses. The broad teachings of the disclosure may be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure may be implemented in and/or combined with features of any of the other embodiments, even if that combination is not explicitly described. In other words, the described embodiments are not mutually exclusive, and permutations of one or more embodiments with one another remain within the scope of this disclosure.

Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein. Also, in the foregoing description, numerous details are set forth to further describe and explain one or more embodiments. These details include system configurations, block module diagrams, flowcharts (including transaction diagrams), and accompanying written description. While these details are helpful to explain one or more embodiments of the disclosure, those skilled in the art will understand that these specific details are not required in order to practice the embodiments.

As will be appreciated by one skilled in the art, aspects of the present disclosure may be embodied as an apparatus that incorporates some software components. Accordingly, some embodiments of the present disclosure, or portions thereof, may combine one or more hardware components such as microprocessors, microcontrollers, or digital sequential logic, etc., such as a processor, or processors, with one or more software components (e.g., program code, firmware, resident software, micro-code, etc.) stored in a tangible computer-readable memory device such as a tangible computer memory device, that in combination form a specifically configured apparatus that performs the functions as described herein. These combinations that form specially-programmed devices may be generally referred to herein as modules. The software component portions of the modules may be written in any computer language and may be a portion of a monolithic code base, or may be developed in more discrete code portions such as is typical in object-oriented computer languages. In addition, the modules may be distributed across a plurality of computer platforms, servers, terminals, mobile devices and the like. A given module may even be implemented such that the described functions are performed by separate processors and/or computing hardware platforms.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

Those of ordinary skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of ordinary skill in the art would further appreciate that the various illustrative logical blocks, modules, circuits, and process steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in various ways for each particular application, but such implementation decisions should not be interpreted as causing a departure from the scope of the present invention.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or process described in connection with the embodiments discloses herein may be embodied directly in hardware, in a software module executed by a processor, or in a combination of the two. A software module may reside in RAM memory, flash memory, ROM memory, EPROM memory, EEPROM memory, registers, hard disk, solid state disk, optical media (e.g., CD-ROM), or any other form of transitory or non-transitory storage medium known in the art. An exemplary storage medium can be coupled to the processor such that the processor can read information from, and write information to, the storage medium. In the alternative, the storage medium can be integral to the processor. The processor and the storage medium may reside in an ASIC. The ASIC may reside in a user terminal. In the alternative, the processor and the storage medium may reside as discrete components in a user terminal.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A device or sensor comprising:
a top asymmetric Au metastructure comprising at least one Au “yin-yang-shaped” nanoantenna disposed on a substrate,
wherein optionally the nanoantenna comprises a biological molecule, and optionally the biological molecule comprises a nucleic acid, a protein, a lipid or a polysaccharide.

2. The device or sensor according to claim 1, further comprising:
a backreflector layer disposed between the top asymmetric Au metastructure and the substrate.

3. The device or sensor according to claim 2 further comprising:
a polymer layer disposed between the top asymmetric Au metastructure and the backreflector layer.

4. The device or sensor according to claim 3, wherein the backreflector layer comprises gold.

5. The device or sensor according to claim 3 wherein the top asymmetric Au metastructure comprises at least two “ying-yang-shaped” nanoantennas separated by a gap, wherein the gap and the nanoantenna radii are optimized using a machine learning algorithm.

6. The device or sensor according to claim 3, wherein the top asymmetric Au metastructure comprises a central nanodisk with encompassing three “yin-yang-shaped” split-ring nanoantennas in clockwise direction wherein the central nanodisk and flanking nanoantenna radii are optimized using a machine learning algorithm.

7. The device or sensor according to claim 6 wherein an initial separation gap d, between the nanodisk and nanoantennas from their centers is optimized to induce maximum interparticle coupling.

8. The device or sensor according to claim 6 wherein the polymer layer comprises PMMA, the backreflector layer comprises gold, and the deposited on a gold coated silicon substrate.

9. A method of using the device or sensor of claim 1, wherein circularly polarized light (CPL) is incident from a first side and reflected and transmitted lights are collected at the first side and an opposing side.

10. A method of fabricating a chiral structure as a plasmonic meta-surface on a transparent substrate using electron beam evaporation and electron beam lithography processes comprising:
preparing a transparent silicon dioxide substrate with a combination of Au/dielectric layers, wherein the structures comprise SiO₂ coated with about 2 nm of chromium for the adhesion of the reflective metallic mirror layer of about 100 nm on a substrate; and
covering an Au layer with about 30 nm Alumina (Al₂O₃), which has a slightly higher refractive index.

11. The method of claim 10 wherein the Au and dielectric layers are deposited using electron beam evaporation.

12. A method for designing and/or optimizing a three-dimensional chiral metamaterial, the method being implemented by a processor and memory, the method comprising:
receiving input geometric design parameters for the chiral metamaterial by an end-to-end functional bi-directional multitask deep-learning (MDL) model comprising a neural network, the MDL model being configured to predict a chiroptical response of the chiral metamaterial via a forward prediction path; and
receiving input for a chiroptical response by the MDL model, the MDL model being further configured to predict geometric design parameters for the chiral metamaterial via an inverse prediction path.

13. The method of claim 12, wherein the MDL model is:
(a) trained using a joint-learning feature based on a joint multitask cost function to enhance prediction accuracy for the forward and/or inverse prediction paths;
(b) configured in the forward prediction path to perform at least a main task and an auxiliary task; and/or
(c) configured to normalize the input geometric design parameters and combine the normalized geometric design parameters with spectral data points.

14. The method of claim 12, wherein the chiral metamaterial comprises at least one “yin-yang-shaped” nanoantenna disposed on a substrate,
wherein the geometric design parameters comprise one or more of a substrate layer thickness, a backreflector layer thickness, a polymer layer thickness, radii of the “yin-yang-shaped” nanoantenna, or a gap between multiple “yin-yang-shaped” nanoantennas, and
wherein the chiroptical response comprises one or more of left circularly polarized (LCP) light absorption, right circularly polarized (RCP) light absorption, or circular dichroism (CD) spectral values.

15. A system for three-dimensional chiral metamaterial design and optimization comprising:
an end-to-end functional bi-directional deep-learning (DL) model implemented by a processor and memory, wherein the model utilizes multitask joint learning features to recognize, generalize and explore a relationship between the metamaterials' geometry and their chiroptical response in both forward and inverse directions,
wherein the model efficiently realizes both forward and inverse retrieval tasks.

16. A chiral metamaterial absorber modeled after the yin-yang symbol comprising:
a top yin-yang shaped Au nanoparticle (YNP),
a PMMA layer,
an Au backreflector; and
a bottom glass layer.

17. A MDL-optimized chiral structure applied in the application of sensing biomolecular enantiomers.

18. A method for detecting a biological molecule of interest in a sample comprising contacting a biological sample with a device or sensor of claim 1, and determining if the biological molecule of interest specifically binds to a nanoantenna of the device or sensor,
wherein optionally the biological molecule of interest is derived or taken from a blood, serum or sputum sample, or a tissue sample, or a biopsy, and optionally the biological molecule of interest is or comprises a nucleic acid, a protein, a lipid or a polysaccharide, and optionally the nucleic acid comprises a DNA, a cDNA or an RNA,
and optionally the nanoantenna comprises or has affixed thereon a biological molecule, and optionally the biological molecule comprises a nucleic acid, a protein, a lipid or a polysaccharide, and optionally the nucleic acid comprises a DNA, a cDNA or an RNA,
optionally the biological molecule of interest is derived or taken from a pathogen, and optionally the pathogen is a bacteria or a virus, and optionally the pathogen is a coronavirus, and optionally the coronavirus is a SARS-2 or COVID-19 virus.

19. The method of claim 18, further comprising diagnosing a disease, infection or condition comprising: contacting a biological sample with a device or sensor of claim 1, and determining if the biological molecule of interest specifically binds to a nanoantenna of the device or sensor, wherein if the biological molecule of interest is detected or determined to be present in the biological sample by the detection of its specific binding to a nanoantenna of the device or sensor, an individual in need thereof from which the biological sample was derived is diagnosed with the disease, condition or infection, wherein optionally the infection is a viral infection, and optionally the viral infection is a coronavirus infection, and optionally the coronavirus infection is a COVID-19 or a SARS-2 infection.

20. The method of claim 19, further comprising treating or ameliorating the disease, infection or condition comprising: administering a treatment or a drug to treat or ameliorate the disease, infection or condition diagnosed or detected in the individual in need thereof.


SEQUENCE LISTING (3 sequences)

SEQ ID NO: 1 - DNA, 25 bp, artificial sequence
  synthetic polynucleotide
gcatctcctgatgaggttccacctg

SEQ ID NO: 2 - DNA, 26 bp, artificial sequence
  synthetic polynucleotide
gcatcaccggatgatgttccacctgg

SEQ ID NO: 3 - DNA, 28 bp, artificial sequence
  synthetic polynucleotide
ccataacctttccacataccgcagacgg